(19) United States
(12) Patent Application Publication
(10) Pub. No.: US 2026/0188485 A1
(43) Pub. Date: Jul. 2, 2026

(54) METHOD AND APPARATUS FOR INTEGRATED HEALTHCARE MANAGEMENT WITH GEOLOCATION CAPABILITIES

(71) Applicant: PERSOWN Analytics, Inc., Jacksonville, FL (US)

(72) Inventor: William D. Meadow, Jacksonville, FL (US)

(73) Assignee: PERSOWN Analytics, Inc., Jacksonville, FL (US)

(21) Appl. No.: 19/437,966

(22) Filed: Dec. 31, 2025

Related U.S. Application Data
(60) Provisional application No. 63/741,093, filed on Jan. 1, 2025.

(51) Int. Cl. G16H 40/67; G16H 10/40; G16H 10/60; G16H 20/00; G16H 30/40; G16H 50/30; G16H 80/00

(52) U.S. Cl. CPC G16H 40/67; G16H 10/40; G16H 10/60; G16H 20/00; G16H 30/40; G16H 50/30; G16H 80/00

(57) ABSTRACT

An integrated system manages real-time healthcare data with geolocation capabilities. A controller operates interfaces to ingest geolocation and to receive patient data from EHRs, remote monitoring, providers, devices, third parties, and applications. A data harmonization module standardizes multimodal inputs; an anonymization/deanonymization service governs personally identifiable information. Secure storage preserves integrity and audited access. A Continuous Analytics Processing System provides file repositories, transforms, working databases, analyzers, and results. A job management subsystem maintains partner, client, event, and log data. A clinical decision support platform generates evidence-based insights, personalized recommendations, and real-time alerts contextually adapted to patient location. An analytics engine performs descriptive, geospatial, network, temporal, predictive, classification, clustering, anomaly detection, NLP, regression, and longitudinal analyses. Deployment spans secure cloud and edge environments with a smart device application. The system produces operational outputs and geolocation-aware interventions to enhance care delivery and infection prevention.

### CROSS REFERENCE TO RELATED APPLICATIONS

This application claims priority to and the benefit of U.S. Provisional Application No. 63/741,093, filed Jan. 1, 2025, and entitled METHOD FOR USING AN INTEGRATED REAL-TIME HEALTHCARE DATA MANAGEMENT SYSTEM WITH GEOLOCATION CAPABILITIES, the entire disclosure of which is incorporated herein by reference.

### TECHNICAL FIELD

The present invention pertains to a system and method that efficiently integrates geolocation technology with one or more clinical decision support platforms and advanced data analytics to enhance healthcare delivery. In response to the evolving healthcare landscape, the integration of real-time geospatial data, such as GPS and Wi-Fi positioning, into a sophisticated clinical decision support platform becomes the cornerstone of this innovation. This fusion empowers healthcare providers to not only make informed decisions based on medical data but also incorporate contextual information derived from a patient's geographical location. The present invention employs advanced data analytics techniques, including AI/ML algorithms and trend analysis, to process extensive datasets, offering precision in healthcare delivery, real-time monitoring, and data-driven insights. By adapting recommendations based on a patient's current location, this present invention enhances personalized and context-aware healthcare interventions, ultimately improving patient outcomes and optimizing resource utilization. The present invention provides integrated solution representing a significant leap forward in addressing the complexities of modern healthcare, promising a future where geolocation-aware clinical decision support transforms healthcare experiences.

### BACKGROUND

Access to affordable health services for a billion underserved families is an ongoing challenge for many areas of the world. In particular, platforms do not exist that are capable of providing low-cost diagnostics and patient centric data for millions of people that enable the patients, health care providers, and/or government entities to have health data available in real time across the globe, to improve care and save lives.

One of the key applications of geolocation in clinical trials is patient recruitment and site selection. Geolocation data allows researchers to identify potential trial participants based on their geographic location, ensuring a diverse and representative study population. This targeted approach streamlines the recruitment process, reduces recruitment costs, and enhances the overall efficiency of clinical trial operations. Geolocation technology contributes to the optimization of site selection for clinical trials. By analyzing geographical data, researchers can identify locations with high concentrations of eligible participants, making it easier to establish trial sites in areas where patient recruitment is likely to be successful. This strategic site selection improves the accessibility of trials for potential participants. In the context of remote and decentralized clinical trials, geolocation enables the monitoring of patients participating from their homes.

### SUMMARY OF THE DISCLOSURE

The present invention provides an intersection of geolocation technology, clinical decision support platforms, and advanced data analytics in the healthcare domain. Traditional healthcare systems often grapple with the challenge of integrating real-world contextual information into clinical decision-making processes. This challenge is particularly evident in scenarios where patient care could benefit from location-specific insights. Recognizing this gap, the present invention sets out to seamlessly merge geolocation technology with a clinical decision support platform and advanced data analytics to bring about a comprehensive solution.

The present invention discloses novel methods and apparatus that combine geolocation, blockchain technology, clinical decision support platforms, and advanced data analytics to revolutionize healthcare data management. Leveraging data from diverse geolocation sources, including GPS trackers, facial recognition, Wi-Fi triangulation, cellular triangulation, Bluetooth beacons, near-field communication, login credentials, and check-ins, along with patient data from Electronic Health Records (EHR), Remote Patient Monitoring, Physician offices, Imaging, Nursing homes, Home care, Clinical labs, Point-of-care devices, Internet of Things (IoT), and smart wearables, the system establishes a comprehensive and secure decentralized ledger on the blockchain.

Integrated systems enhance security, transparency, and interoperability, paving the way for personalized healthcare delivery, efficient clinical decision-making, and improved patient outcomes. The method involves securely collecting and storing geolocation data on a blockchain-based decentralized ledger, ensuring data integrity and patient privacy through cryptographic encryption. Smart contracts automate data sharing agreements, enabling controlled access to geolocation and healthcare data among authorized entities. The technology integrates advanced data analytics to provide real-time insights, personalized recommendations, and alerts to healthcare providers based on geolocation patterns and clinical data. The system fosters patient-centric healthcare by allowing individuals to manage consent for sharing specific geolocation information. The inventive method finds applications in clinical trials, pharmaceutical supply chain management, fraud prevention in healthcare billing, and proactive outbreak control. This invention represents a comprehensive approach to leveraging geolocation, blockchain, clinical decision support, and advanced data analytics for a transformative impact on healthcare data management and delivery.

Geolocation technology forms a foundational layer for the systems of the present invention. Leveraging the precision wireless location technologies, such as, for example, one or more of: GPS; Wi-Fi positioning; Bluetooth; Ultra-Wideband, cellular triangulation/trilateration; and other location-based services, the present invention enables accurate determination, tracking, and monitoring of geographical coordinates that may be associated with patients, healthcare assets, treatment centers, therapeutic agent (including pharmaceutical) dispensing facilities, and/or resources in real-time.

Such geospatial data serves as a helpful element in enhancing an overall clinical decision support process by introducing a spatial dimension to medical information. The geospatial data may also be chronologically (or other time related records) related to treatment information or other medically related information. A clinical decision support platform, according to the present invention, integrates technology into a sophisticated system designed to assist healthcare professionals in making informed decisions, allows patients to have access to their health-related data over time and across geopolitical areas.

The present invention combines AI/ML algorithms, comprehensive medical knowledge bases, and patient-specific data to generate evidence-based insights. By incorporating geolocation data, the platform gains the ability to adapt its recommendations based on the patient's current location. This dynamic integration ensures that clinical decisions are not only rooted in medical evidence but are also tailored to the unique circumstances of the patient's geographical context.

Advanced data analytics components of the invention if functional to extract actionable insights from vast datasets generated by clinical and/or geolocation sources. Predictive modeling, trend analysis, and anomaly detection algorithms are deployed to identify meaningful patterns and correlations within the data. Analytical prowess of the present invention supports both individualized patient care and broader population health management strategies by identifying geographical health disparities, optimizing resource allocation, and facilitating proactive healthcare interventions.

As such, the technology provides a holistic approach to addressing challenges experienced by contemporary state of the art healthcare systems. The present invention relatively seamless integration of geolocation technology, clinical decision support platforms, and advanced data analytics creates a synergy that not only enhances the precision and effectiveness of clinical decisions but also opens avenues for personalized, context-aware healthcare interventions. This comprehensive solution marks a significant advancement in the field, promising to elevate the standard of healthcare delivery through the strategic fusion of geospatial intelligence and clinical decision support.

Geolocation technology has emerged as a transformative force in the realm of patient tracking and monitoring within healthcare settings. Innovative processes referencing location-based services are utilized to revolutionize how healthcare providers manage and respond to patient needs in real time.

One primary application lies in Real-Time Location Services (RTLS), where geolocation is employed to precisely track the movement of patients within healthcare facilities. Wearable devices, tags, or mobile applications equipped with geolocation capabilities allow healthcare providers to monitor and navigate patients efficiently, improving overall patient care and streamlining hospital operations. In cases where patient safety is paramount, geolocation technology facilitates the prevention of wandering among individuals with cognitive impairments such as Alzheimer's or dementia. Wearable devices or tags with geolocation capabilities serve as safeguards, triggering alerts for healthcare staff if a patient moves outside predefined safe areas. This not only enhances patient security but also mitigates potential risks associated with unmonitored movement.

Geolocation technology extends its impact to emergency response scenarios, where it plays a helpful role in optimizing ambulance routes. By providing real-time information on traffic conditions and the patient's location, emergency medical services (EMS) can determine the most efficient route for a timely response. Additionally, geolocation facilitates seamless coordination between EMS and hospital emergency departments, allowing healthcare facilities to prepare adequately for incoming patients and optimize emergency care delivery. In the realm of remote patient monitoring, geolocation is a key enabler. Particularly in home-based settings, healthcare providers leverage geolocation-enabled monitoring devices to track patients with chronic conditions. This approach not only allows for proactive healthcare management but also reduces the necessity for frequent hospital visits. Geolocation-enabled devices can trigger alerts when patients move outside predefined geographic areas or when vital signs fall outside normal ranges, ensuring swift responses to potential health issues. Patient flow and capacity management within healthcare facilities are also significantly enhanced through geolocation technology.

By monitoring the movement of patients throughout their care journey, healthcare providers can optimize patient flow, identify operational bottlenecks, reduce wait times, and improve overall efficiency. Furthermore, geolocation data contributes to effective capacity planning, offering insights into the utilization of different areas within a healthcare facility and enabling resource allocation based on real-time needs. An additional layer of patient safety is achieved through the use of geofencing technology, creating virtual boundaries around secure or restricted areas within healthcare facilities. This ensures that only authorized personnel have access to specific locations, contributing to a secure and controlled healthcare environment. In conclusion, the integration of geolocation technology in patient tracking and monitoring represents a paradigm shift in healthcare delivery. It not only improves the quality of care by providing real-time insights but also enhances patient safety, streamlines operational workflows, and contributes to a more efficient and responsive healthcare ecosystem. As technology continues to advance, the integration of geolocation solutions is poised to play an increasingly pivotal role in patient-centered care and healthcare operational excellence.

Wireless geolocation apparatus and processes provide components in optimizing emergency response and ambulance routing, introducing a paradigm shift in the efficiency and effectiveness of emergency medical services (EMS). At the core of this transformation is the ability of geolocation systems to precisely identify the location of emergency incidents, laying the foundation for a well-coordinated and rapid response.

In some embodiments, a degree of the processing as described herein may be performed on a controller, which may include a cloud server, a standalone computing device or a smart device. In many examples, the input file may be communicated by the smart device to a controller embodied in a remote server. In some embodiments, the remote server, which is preferably a cloud server, may have significant computing resources that may be applied to AI algorithmic calculations analyzing the image.

In some embodiments, dedicated integrated circuits tailored for deep learning AI calculations (AI Chips and software algorithms) may be utilized within a controller or in concert with a controller. Dedicated AI chips may be located on a controller, such as a server that supports a cloud service or a local setting directly. For example, a dedicated AI chip may expedite the processing of large-scale architectural plans, enabling quicker turnaround times for alignment reports for defining geofencing areas.

In some embodiments, an AI chip tailored to a particular artificial intelligence AI or machine learning ML (AI/ML) algorithms and/or calculations may be configured into a case that may be connected to a smart device in a wired or wireless manner and may perform a deep learning AI calculation. Such AI chips may be configurable to match a number of hidden levels to be connected, the manner of connection, and physical parameters that correspond to the weighting factors of the connection in the AI engine (sometimes referred to herein as an AI model). In other examples, software-only embodiments of the AI engine may be run on one or more of: local computers, cloud servers, or on smart device processing environments.

Key benefits brought about by the present invention's use of geolocation include the provision of real-time traffic information as it relates to the provision of healthcare. By incorporating dynamic traffic updates, emergency responders can navigate through congested areas with agility, ensuring that ambulances take the fastest and most accessible routes to reach the scene. This feature is particularly helpful in urban settings where traffic conditions can significantly impact response times. Geolocation technology is helpful in determining the quickest route for ambulances to reach the emergency location. Advanced algorithms analyze real-time data, considering factors such as traffic conditions, road closures, and the urgency of the situation. This intelligent routing ensures that ambulances reach their destinations swiftly, minimizing response times and potentially saving lives.

Seamless coordination between EMS and hospital emergency departments is facilitated by geolocation. Through real-time updates on estimated arrival times, patient conditions, and other critical details, hospitals can proactively prepare for incoming patients. This integration enhances overall healthcare delivery by optimizing the allocation of resources and streamlining the transition of patients from the ambulance to the hospital. Mobile applications equipped with geolocation capabilities play a helpful role in emergency reporting. Individuals can report emergencies and share their precise locations through these applications, enabling faster and more accurate responses from emergency services. This feature is particularly valuable in situations where rapid intervention is essential, such as cardiac emergencies or accidents.

Intelligent ambulance dispatch systems leverage geolocation to optimize ambulance deployment. These systems consider multiple variables, including ambulance proximity, traffic conditions, and the severity of the emergency. By dynamically dispatching the closest available unit, these systems ensure an efficient allocation of resources and a rapid response to emergency situations. Within ambulances, geolocation technology is integrated into mobile data terminals, providing real-time navigation assistance to emergency responders. These terminals display optimal routes, traffic conditions, and other critical information, aiding ambulance crews in navigating effectively and safely to the scene. Continuous monitoring of the ambulance's location during transit ensures ongoing coordination between emergency responders and the receiving hospital. The integration of geolocation with Public Safety Answering Points (PSAPs) enhances the accuracy of emergency caller location identification. This information is pivotal for dispatching ambulances to the correct location promptly, contributing to the precision of the emergency response process. In these and other ways, geolocation technology may be used to assist emergency response and ambulance routing by introducing a level of precision and efficiency that was previously unattainable. The ability to identify incident locations, optimize routes, and coordinate seamlessly with emergency departments translates into reduced response times, improved patient outcomes, and an overall enhancement of emergency medical services. As technology continues to advance, geolocation is poised to play an increasingly pivotal role in shaping the future of emergency response systems.

Geolocation technology has emerged as a cornerstone in revolutionizing asset and inventory management within healthcare environments. This innovative integration provides healthcare facilities with the capability to streamline operations, optimize resource utilization, and ensure the efficient tracking of critical assets. At the forefront of geolocation's impact is its ability to facilitate real-time tracking of medical equipment. Through the deployment of geolocation tags or sensors on each piece of equipment, healthcare staff can seamlessly monitor the location and status of assets. This feature is particularly helpful in large healthcare settings where the efficient utilization of medical equipment significantly influences patient care outcomes. A key application of geolocation in asset management is the creation of a location-based inventory control system. Geolocation technology enables healthcare facilities to track the movement of inventory within specific areas, optimizing storage and retrieval processes. This ensures that medical supplies are strategically placed for easy accessibility, contributing to the overall efficiency of inventory management.

Geolocation technology acts as a preventative measure against the loss or misplacement of assets and inventory items. The real-time tracking capability allows staff to swiftly identify the last known location of an asset, reducing the time spent searching for misplaced items and minimizing the risk of loss. This enhances operational efficiency and ensures that resources are utilized optimally. Insights into the utilization patterns of equipment are made possible through geolocation data. This information empowers healthcare facilities to make informed decisions regarding equipment distribution, maintenance schedules, and resource allocation. The result is the optimization of asset utilization, reducing downtime and enhancing overall operational effectiveness. The integration of geolocation into automated inventory management systems proves invaluable for timely inventory replenishment. By triggering alerts for low-stock items based on their geographical location, healthcare facilities can minimize stockouts, ensure continuous availability of critical supplies, and contribute to the overall efficiency of inventory management processes.

Geolocation technology serves as a security-enhancing mechanism by providing real-time information on the location of high-value assets. This not only acts as a deterrent against theft but also facilitates the swift recovery of assets in the event of unauthorized removal. The security measures bolstered by geolocation contribute to maintaining a secure and controlled healthcare environment. Geofencing technology is leveraged to create virtual boundaries around restricted areas where certain assets are stored. This ensures that access to these areas is limited to authorized personnel, and alerts can be triggered if assets equipped with geolocation enter or exit these predefined zones.

Geofencing adds an extra layer of security to the management of critical assets. In addition to tracking the location of assets, geolocation-enabled sensors can be integrated with environmental monitoring systems. This is particularly relevant for assets and inventory items that require specific temperature conditions. Geolocation data provides additional contextual information, ensuring that environmental parameters are maintained during transportation and storage. Geolocation simplifies the auditing process by offering a real-time snapshot of asset and inventory locations. This streamlines compliance efforts, making it easier for healthcare facilities to adhere to regulatory requirements and conduct accurate audits. Geolocation's role in audits contributes to maintaining compliance with industry standards and regulations. The integration of geolocation with maintenance management systems is helpful in scheduling preventive maintenance based on equipment location and usage patterns.

Such proactive approaches to maintenance helps extend a lifespan of assets, reduce unplanned downtime, and ensure that critical equipment remains in optimal working condition. In conclusion, the utilization of geolocation in asset and inventory management signifies a transformative shift in healthcare operations. The real-time tracking, optimization of inventory control, and enhanced security measures contribute to operational efficiency, cost savings, and an elevated standard of patient care. As geolocation technology continues to evolve, its applications in asset and inventory management are poised to advance further optimizing resource utilization in healthcare settings.

Geofencing technology, when applied to patient safety in healthcare settings, introduces a highly effective and personalized approach to secure and monitor individuals, particularly those at risk due to cognitive impairments. Geofencing creates virtual boundaries or safe zones within healthcare facilities, ensuring that patients remain within predefined areas for their safety. This technology is particularly valuable for individuals with conditions such as Alzheimer's or dementia, where wandering behavior can pose significant risks. The primary objective of geofencing for patient safety is to prevent wandering among individuals with cognitive impairments.

Wearable devices or tags equipped with geolocation capabilities are provided to patients, enabling healthcare staff to define virtual boundaries around secure or designated areas within the facility. If a patient wearing such a device approaches or crosses the predefined boundary, geofencing triggers alerts to healthcare staff, ensuring timely intervention to prevent potential safety issues. The customization and adaptability of geofencing technology are helpful aspects of its application in patient safety. Virtual boundaries can be tailored to each patient's specific needs and the layout of the healthcare facility. This level of customization allows healthcare providers to address individual patient requirements, ensuring that alerts are triggered only when patients move outside predetermined safe areas, providing a balance between safety and independence. Geofencing also plays a role in creating a secure environment for patients during specific medical procedures or examinations.

Virtual boundaries can be established around restricted areas, such as operating rooms or sensitive diagnostic areas, ensuring that only authorized personnel and patients within specific conditions have access. Such applications enhance the overall safety and security of patients undergoing medical treatments. In emergency situations, geofencing contributes to the rapid response and location tracking of patients. If a patient with a geolocation-enabled device requires immediate assistance or experiences a medical emergency, healthcare providers can quickly pinpoint the patient's location within the facility. This information is vital for expediting emergency response times and providing timely and targeted care.

Continuous monitoring capabilities of geofencing technology enhance patient safety by keeping healthcare staff informed of patients' movements in real-time. This is particularly valuable during shifts where staffing levels may be lower, ensuring that even with reduced personnel, patient safety is maintained. The technology acts as an additional layer of vigilance, contributing to a proactive and responsive healthcare environment. Privacy considerations may be included in some implementations of geofencing for patient safety.

Healthcare providers must establish transparent communication with patients and their families, explaining the purpose and benefits of geofencing. Clear guidelines on how location data is used, stored, and secured should be established to ensure compliance with privacy regulations and to build trust among patients and their caregivers. In conclusion, geofencing for patient safety harnesses the power of geolocation technology to create a secure and personalized environment within healthcare facilities. The tailored virtual boundaries, emergency response capabilities, and continuous monitoring contribute to mitigating safety risks for patients with cognitive impairments.

Geolocation technology plays a pivotal role in advancing population health management strategies by providing valuable insights into the geographical distribution of health-related data. This innovative approach leverages location-based information to analyze and address health disparities, improve healthcare resource allocation, and enhance overall population health outcomes. One of the primary applications of geolocation in population health management includes the analysis of geospatial health data. This involves mapping health-related data points, such as disease prevalence, healthcare facility locations, and demographic information, onto geographical maps. By visually representing these data points, healthcare providers and public health officials can identify patterns, trends, and disparities in health outcomes across different geographic regions. Geolocation technology enables the identification of health disparities and social determinants of health based on geographical data.

Understanding a distribution of healthcare resources, socioeconomic factors, and environmental conditions allows healthcare organizations to tailor interventions to address specific needs within different communities. Such targeted approaches contribute to reducing health inequities and improving overall population health. In the context of infectious disease management, geolocation is helpful in tracking and monitoring disease outbreaks.

Utilizing mapping of a geographic spread of diseases, public health authorities, or other relevant entities may implement timely and targeted interventions, allocate resources effectively, and implement preventive measures in high-risk areas.

Geospatial analysis may be used to enhance a capacity to contain and manage the spread of infectious diseases within populations. Geolocation supports the optimization of healthcare resource allocation by identifying areas with higher healthcare needs. By mapping the locations of healthcare facilities, providers, and community resources, decision-makers can strategically allocate resources to areas with higher prevalence rates or specific health challenges. This approach ensures that healthcare services are distributed efficiently, improving accessibility for the population.

Integration of geolocation in telehealth and mobile health applications enhances population health management by facilitating remote patient monitoring and intervention. Geolocation-enabled mobile apps can track patient locations, enabling healthcare providers to monitor adherence to treatment plans, provide targeted health education, and intervene proactively based on geographic-specific health risks. Geofencing technology is employed to create virtual boundaries around specific areas, triggering alerts or interventions when individuals enter or exit predefined zones. This can be utilized for population health management initiatives, such as promoting healthier behaviors or encouraging preventive screenings.

Geofencing contributes to personalized interventions based on the geographic context of individuals' daily lives. In disaster preparedness and response, geolocation facilitates the management of population health during emergencies. Mapping vulnerable populations, evacuation routes, and healthcare facility locations enables efficient emergency response planning. Geolocation data assists in coordinating evacuation efforts, deploying medical resources, and ensuring the safety and well-being of affected populations. Privacy considerations are paramount in geolocation-based population health management initiatives. Healthcare organizations must implement robust data security measures, anonymize data where appropriate, and adhere to relevant privacy regulations. Transparent communication with individuals about the use of geolocation data ensures trust and compliance with privacy standards. In conclusion, the use of geolocation in population health management represents a transformative approach to understanding and addressing health challenges within communities. By harnessing the power of geospatial data, healthcare organizations can tailor interventions, allocate resources effectively, and enhance the overall health and well-being of diverse populations. As technology continues to evolve, geolocation is expected to play an increasingly vital role in shaping the future of population health management strategies.

Geolocation technology is used in some implementations, such as those that include one or more of: telemedicine and remote patient monitoring (RPM) initiatives, revolutionizing the way healthcare is delivered beyond traditional clinical settings. The innovative application of geolocation presented by the present invention enhances the capabilities of telehealth platforms and RPM systems, and enables improved patient care, proactive monitoring, and more efficient coordination of virtual healthcare services than previously available.

In some implementations that include telemedicine, geolocation technology is utilized for tracking and monitoring patients in their homes. Geolocation-enabled devices, such as smartphones or wearables, allow healthcare providers to gather real-time location data, ensuring that patients are within suitable locations with a stable internet connection for virtual consultations. This feature streamlines the coordination of telehealth appointments, enhancing the overall accessibility and effectiveness of remote healthcare services. Remote patient monitoring benefits significantly from geolocation technology, particularly in chronic disease management. Geolocation-enabled devices can track patients' movements, providing insights into their daily activities and routines.

Geolocation data may be processed for assessing patient behavior, adherence to treatment plans, and identifying potential health risks. Geolocation enhances the granularity of monitoring, allowing healthcare providers to tailor interventions based on the patient's geographic context. Geolocation supports the concept of “geo-fencing” in remote patient monitoring, creating virtual boundaries around specific geographic areas. This feature enables healthcare providers to set parameters for patients' movement, triggering alerts if patients venture outside predefined safe zones. In cases where patients with certain conditions require restricted mobility or are at risk of wandering, geofencing ensures timely interventions and enhances patient safety.

Integration of geolocation with telemedicine platforms facilitates the coordination of virtual healthcare services in emergencies. During telehealth appointments, geolocation data ensures that patients can access care from appropriate locations. In emergency situations, such as sudden health deteriorations, healthcare providers can quickly identify the patient's location for targeted interventions or dispatching emergency services. Geolocation technology contributes to the continuous monitoring of patients in real time, enabling healthcare providers to intervene proactively based on their geographic location.

For example, if a patient with a chronic condition experiences a significant change in location or is outside predefined boundaries, healthcare providers can receive alerts and initiate appropriate interventions to prevent potential health complications. In mental health telemedicine, geolocation plays a role in understanding the environmental factors that may impact a patient's well-being. Analyzing geospatial data helps healthcare providers assess factors such as access to social support, community resources, and potential stressors in the patient's environment. This holistic approach enhances the personalized nature of mental health interventions delivered through telemedicine. Privacy considerations are paramount in the use of geolocation for telemedicine and remote patient monitoring. Healthcare organizations must implement robust security measures to protect patient data, anonymize location information when appropriate, and adhere to relevant privacy regulations. Transparent communication with patients about the use of geolocation data is essential to build trust and ensure compliance with privacy standards. In conclusion, the integration of geolocation technology in telemedicine and remote patient monitoring represents a transformative shift in healthcare delivery. By harnessing real-time location data, healthcare providers can enhance the accessibility, personalization, and proactive nature of virtual care. As technology continues to advance, geolocation is poised to play an increasingly vital role in shaping the future of telehealth and remote patient monitoring strategies.

According to the present invention, geolocation technology provides a powerful tool in the realm of clinical trials and research, offering innovative solutions to enhance the efficiency, accuracy, and patient-centric nature of these endeavors. The integration of geolocation adds a spatial dimension to clinical trial activities, providing valuable insights into patient recruitment, monitoring, and data collection.

Wearable devices equipped with geolocation capabilities provide real-time location data, allowing researchers to track patients' movements and adherence to trial protocols. This enhances the quality of data collected and ensures compliance with trial requirements. Geolocation plays a pivotal role in ensuring patient safety and compliance during clinical trials. Virtual boundaries or geofencing can be established around trial sites or specific geographic areas to trigger alerts if participants enter or exit predefined zones. This feature is particularly valuable in ensuring that patients follow trial protocols and visit designated locations for assessments. The use of geolocation in clinical trials extends to the monitoring of environmental factors that may impact study outcomes. Researchers can analyze geospatial data to assess environmental variables such as air quality, climate, or proximity to healthcare facilities. This information adds a contextual layer to research findings, allowing for a more comprehensive understanding of the study population.

Geolocation technology facilitates the collection of real-world evidence by capturing data on participants' daily activities and movements. This information is valuable for assessing the impact of interventions in real-life settings, providing insights into how treatments or therapies affect patients in their natural environments. Geolocation adds a dynamic and contextual dimension to the evidence generated in clinical trials. In multinational or multicenter clinical trials, geolocation assists in coordinating activities across diverse geographic locations. Researchers can track the progress of trial activities, monitor site performance, and ensure consistent data collection standards. This enhances the overall management and coordination of complex clinical trials conducted in different regions. Privacy considerations may be an important aspect for the use of geolocation for clinical trials and research. Researchers must implement robust security measures to protect participants' location data, anonymize information when appropriate, and adhere to ethical and regulatory standards. Transparent communication with participants about the use of geolocation data is crucial to ensure trust and compliance. In conclusion, the integration of geolocation technology in clinical trials and research represents a paradigm shift in the way studies are designed, conducted, and monitored. By leveraging spatial data, researchers can enhance patient recruitment, improve monitoring capabilities, and capture a more holistic view of study participants. As technology continues to advance, geolocation is poised to play an increasingly integral role in shaping the future of clinical trials and contributing to the generation of robust and patient-centric research outcomes.

The present invention provides a comprehensive, cloud-based Healthcare Intelligence and Operational Platform designed to synchronize disparate data streams into actionable clinical and logistical insights. By integrating high-fidelity geolocation data with longitudinal health records and real-time environmental monitoring, the invention bridges the gap between individual patient care, facility management, and public health surveillance.

Core Pillars of the Platform may include one or more of: a) Multi-Dimensional Data Fusion: The system leverages a sophisticated technology stack to harmonize data from traditional sources (EHRs, Lab results) with emerging Internet of Things (IoT) and smart wearable metrics. This includes specialized biosensors in clothing, eyewear, and contact lenses, providing a continuous “biological snapshot” of the patient; b) Spatial and Temporal Intelligence: Unlike traditional static records, this invention treats location and time as primary diagnostic variables. By utilizing Wi-Fi/Cellular triangulation and GPS, the platform identifies “hotspots” of infection and predicts disease spread patterns before they manifest as clinical crises; c) Decentralized Security and Trust: Through the integration of Blockchain technology, the invention ensures that sensitive healthcare data remains immutable and transparent. Smart contracts automate data-sharing agreements, while decentralized identity management returns control of health information to the patient, ensuring strict compliance with global privacy regulations (e.g., HIPAA, GDPR); and Immersive Clinical Interaction: The platform incorporates Augmented and Virtual Reality (AR/VR) as a primary interface for data collection and consultation. This allows for spatial documentation of patient encounters and provides clinicians with a 3D environment to visualize complex diagnostic data and collaborate remotely in real-time.

The versatility of the architecture of the present invention allows it to be deployed across several critical healthcare domains such as, for example, but not limited to, or more of the following: a) Infection Control & Public Health: continuous technology stacking identifies potential outbreaks and automates Antimicrobial Resistance (AMR) surveillance, helping providers practice better antibiotic stewardship; b) Emergency Response Optimization: the system transforms ambulance dispatch from a reactive service to a proactive one by predicting high-risk zones and optimizing vehicle staging based on real-time geospatial and temporal trends; c) Facility & Environmental Management: the platform extends into the physical environment, using AI to detect microbial threats in hospital rooms and automatically dispatching disinfection crews with pathogen-specific protocols and products; and d) Pharmaceutical Integrity: by linking the drug supply chain to the blockchain with geolocation markers, the invention provides end-to-end traceability, effectively eliminating the risk of counterfeit medications.

Generally, the invention represents a shift toward “Learning Health Systems.” Through continuous AI/ML model refinement and interoperability with external datasets, the platform evolves with every data point collected, resulting in a self-optimizing ecosystem that improves patient outcomes, enhances provider efficiency, and secures the global healthcare supply chain.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 illustrates a schematic diagram of an exemplary automated platform according to some embodiments of the present invention.

FIG. 2 illustrates a schematic diagram of an exemplary automated platform according to some embodiments of the present invention with CAPS aspects expounded upon.

FIG. 3 illustrates a schematic diagram of an exemplary automated platform according to some embodiments of the present invention with Transform aspects expounded upon.

FIG. 4 illustrates a schematic diagram of an exemplary automated controllers according to some embodiments of the present invention with SAS Viya expounded upon.

FIG. 5 illustrates a schematic diagram of an exemplary controller according to some embodiments of the present invention.

FIG. 6 illustrates a flowchart with method steps that may be implemented in some embodiments of the present invention.

FIGS. 7-7A are a flowchart illustrating a method for healthcare data analytics and infection control.

FIG. 8 is a flowchart illustrating a method for emergency medical response and ambulance optimization.

FIG. 9 is a flowchart illustrating a method for healthcare asset and inventory management.

FIG. 10 is a flowchart illustrating a method for longitudinal health data analysis and treatment assessment.

FIG. 11 is a flowchart illustrating a method for cloud-based ambulance deployment optimization.

FIG. 12 is a flowchart illustrating a method for geolocation-based clinical trial research and data management.

FIG. 13 is a flowchart illustrating a method for a clinical decision support system for infectious disease and antibiotic stewardship.

FIG. 14 is a flowchart illustrating a method for automated microbial infection response and facility disinfection.

FIG. 15 is a flowchart illustrating a method for blockchain-based healthcare data management and security.

FIG. 16 is a flowchart illustrating a method for integrated healthcare data analytics using a technology stack.

### DETAILED DESCRIPTION

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

The present invention discloses methods and systems for infection control that leverage geolocation data from a diverse array of sources. Automated apparatus may receive geolocation data from GPS trackers, facial recognition, Wi-Fi triangulation, cellular triangulation, Bluetooth beacons, near-field communication, ultra-wideband enabled devices, login credentials, and check-ins. Automated systems that use a variety of common programming libraries can be easily configured to harmonize patient data from Electronic Health Record (EHR) data from various sources, including acute care sites, Remote Patient Monitoring, Physician offices, Imaging, Nursing homes, Home care, Clinical labs, Point-of-care devices, Internet of Things (IoT), and smart wearables so that formats and meanings of data values can match in common way for clinically relevant use and analytics. Utilizing retrospective Domestic and International data enhances artificial intelligence/machine learning (AI/ML) training and longitudinal data analysis. Descriptive analytics are applied to understand data distribution, patterns, and trends, while geospatial analysis identifies spatial patterns and hotspots related to patient movement. Facial recognition enhances security and monitors patient interactions.

Network analysis examines relationships using Bluetooth beacons, NFC, and Wi-Fi triangulation. Temporal analysis tracks patient movements and monitors changes over time. Predictive analytics forecast disease outbreaks and predict patient health deterioration. AI/ML is used for classification, clustering, and anomaly detection. Natural language processing extracts insights from textual data, and regression analysis predicts outcomes. Continuous technology stacking analyzes harmonized data for potential infection outbreaks, guiding the implementation of infection control measures. This method offers a holistic and advanced approach to infection control, contributing significantly to the prevention and containment of infectious diseases.

The automated systems of the present invention provide synergies amongst geolocation technology, a clinical decision support platform, and advanced data analytics holds immense potential in revolutionizing chronic disease management. Geolocation data provides a dynamic and contextual understanding of a patient's environment, enabling healthcare professionals to tailor interventions based on real-time information. This integration becomes particularly valuable in chronic disease management, where lifestyle factors and environmental influences play a crucial role.

### Glossary

For the purposes of this disclosure, the glossary terms provided herein are intended to facilitate understanding of the present subject matter and its embodiments. These definitions are inclusive and provided for illustrative purposes, and are not intended to be limiting or comprehensive unless expressly stated otherwise in the claims. The meanings ascribed to the glossary terms are to be interpreted in a reasonable manner consistent with the context in which they are used, and are intended to encompass all technical and functional equivalents that would be understood by those skilled in the art. Where a term is defined in the claims, the definition in the claims will govern. Otherwise, the glossary terms are provided to clarify the description and are not to be construed as restricting the scope of the subject matter or its various embodiments. The use of a particular term in the singular or plural, or with or without capitalization, is not intended to be limiting unless the context clearly indicates otherwise.

Adaptive Boosting (AdaBoost)—An ensemble learning algorithm that combines predictions from multiple weak learners to improve classification accuracy.

Advanced Data Analytics—The use of techniques such as predictive modeling, trend analysis, anomaly detection, and geospatial analysis to extract actionable insights from large datasets.

AI Training Component—A system or module used to train machine learning models using historical and real-time data supplemented with artificial intelligence tools that provide interaction with people.

Anomaly Detection—The identification of unusual patterns or outliers in data, which may indicate potential issues such as abnormal patient behavior or security breaches.

Anomaly Detection Algorithms—Techniques used to identify unusual patterns or outliers in data, indicating potential issues or risks.

Antibiotic Stewardship—A set of practices aimed at optimizing the use of antibiotics to combat antimicrobial resistance and improve patient outcomes.

Antimicrobial Resistance (AMR)—The ability of pathogens to resist the effects of antibiotics, posing challenges to infection control and treatment.

ARIMA—A statistical model used for time-series forecasting, accounting for trends and seasonality in data.

Asset and Inventory Management—The use of geolocation, asset identification technologies and sometimes blockchain for proof of provenance to track, optimize, and secure healthcare resources and equipment.

Artificial Intelligence (AI)—The simulation of human intelligence by machines, including processors configured to perform capabilities including, but not limited to: decision-making, pattern recognition, and natural language understanding.

AI Agent—An autonomous software module configured to cause a processor to resolve conflicts between functional requirements without human intervention by applying one or both of: (a) a user-defined hierarchy of priorities and (b) a learned hierarchy of priorities derived from historical project data and feedback loops.

Augmented Reality (AR)—A technology that overlays digital information onto the real-world environment, enhancing visualization and interaction for applications such as surgical planning and patient education.

Healthcare Data Analytics—The examination of extensive and intricate healthcare datasets to generate insights aimed at enhancing patient care, operational efficiency, and research outcomes.

Blockchain—A decentralized and immutable ledger technology used for securely storing and sharing data, ensuring transparency, data integrity, and cryptographic encryption.

Blockchain-Based Decentralized Ledger—A secure and transparent system for storing healthcare data, ensuring data integrity and patient privacy through cryptographic encryption.

Chronic Disease Management—Strategies for monitoring and treating long-term health conditions using personalized care plans and real-time data analytics.

Clinical Decision Support Platform (CDSP)—A sophisticated system designed to assist healthcare professionals in making informed decisions by providing evidence-based insights, treatment recommendations, and patient-specific guidelines.

Controller—A computing device, such as a cloud server or mobile device, configured to execute processes such as but not limited to geolocation, data analytics, and clinical decision support.

Data Harmonization Module—A system component that integrates and standardizes data from diverse sources with different formats, values, and accuracies for consistent and meaningful analysis.

Data Privacy Regulations—Legal frameworks such as HIPAA and GDPR that govern the protection and confidentiality of patient data.

Data Visualization—The graphical representation of data to enhance understanding and support decision-making.

Deep Q-Network (DQN)—A reinforcement learning algorithm that uses deep neural networks to optimize decision-making processes.

Electronic Health Records (EHR)—Digital records of patient health information, including complete medical history, diagnoses, treatment plans, environment and test results, used for clinical decision-making and data harmonization.

Emergency Response Optimization—The use of geolocation and real-time data to improve response by personnel and equipment such as ambulance deployment and emergency care delivery.

Extract, Transform, Load (ETL)—A process used to clean, integrate, and transform data from various sources into a standardized format for analysis.

Facility Cleaning Protocols—Guidelines generated based on microbial data analysis to ensure effective cleaning and disinfection in healthcare environments.

Facial Recognition—A biometric technology that identifies individuals based on facial features, used for security and monitoring purposes in healthcare settings.

Geofencing—A technology that creates virtual boundaries around specific geographic areas, triggering alerts or actions when an individual or object enters or exits these zones.

Geolocation—The process of identifying the geographical location of an object or individual using technologies such as Satellites, GPS, Wi-Fi triangulation, Bluetooth beacons, cellular triangulation, and near-field communication.

Geolocation Module—A component of the system responsible for collecting and processing geolocation data from various sources.

Geolocation Patterns—Trends and correlations identified in geolocation data, used for healthcare decision-making and resource optimization.

Geolocation Tracking—The continuous monitoring of an individual's location using geolocation-enabled devices and technologies.

Geolocation-Enabled Wearables—Smart devices such as rings, shirts, belts, glasses, and contacts equipped with geolocation capabilities for tracking and monitoring health metrics.

Geospatial Analysis—The examination of geographical data to identify spatial patterns, trends, and correlations, often used in healthcare for resource allocation and disease tracking.

Geospatial Health Data—Health-related data mapped onto geographical locations to analyze patterns, trends, and disparities in health outcomes.

Healthcare Data Analytics—The process of analyzing healthcare data to improve patient outcomes, operational efficiency, and research.

Hoeffding Tree—An online decision tree algorithm designed for real-time classification tasks in data streams, adapting to evolving patterns and data changes.

Immersive Visualizations—The use of AR and VR technologies to create engaging and detailed representations of patient data for enhanced understanding and decision-making.

Interoperability—The ability of different systems and technologies to work together seamlessly, enabling efficient data exchange and integration.

Internet of Things (IoT)—A network of interconnected devices, such as smart wearables and medical equipment, which collect and exchange data to enhance healthcare delivery and monitoring.

Isolation Forest—An anomaly detection algorithm that isolates anomalies by partitioning data into small clusters.

Longitudinal Data Analysis—The study of data collected over time to identify trends, assess treatment effectiveness, and support evidence-based healthcare decisions.

Machine Learned Models—AI/ML models developed through training on datasets to perform specific tasks such as classification, prediction, or anomaly detection.

Machine Learning (ML)—A subset of AI that enables systems to learn and improve from data without explicit programming, used for tasks such as classification, clustering, and predictive modeling.

Natural Language Processing (NLP)—A branch of AI that focuses on the interaction between computers and human language, used to extract insights from textual data such as clinician notes and patient communications.

Natural Language Understanding (NLU)—A subset of NLP focused on interpreting and understanding human language in a meaningful way.

Nephrological Conditions—Medical conditions related to kidney health, including chronic kidney disease and renal replacement therapy.

Patient Consent Management—A system that allows patients to control and manage their consent for sharing geolocation and health data securely.

Population Health Management—Strategies and practices aimed at improving the health outcomes of a group or community by addressing health disparities and optimizing resource allocation.

Population Health Trends—Patterns and insights derived from analyzing health data across communities to inform public health strategies.

Post-Surgical Monitoring—The tracking of patient recovery and potential complications following surgery using real-time data, analytics, and reporting.

Predictive Analytics—The application of AI/ML algorithms to forecast future events or trends based on historical and real-time data.

Predictive Modeling—The use of statistical and machine learning techniques to forecast future events or trends based on historical and real-time data.

Prophet—A forecasting model developed by Facebook, designed for time-series data with daily observations and patterns on different time scales.

Real-Time Alerts—Notifications generated based on real-time data analysis to inform healthcare providers of significant events or conditions.

Real-Time Clinical Decision Platforms—Systems that integrate geolocation, patient data, and advanced analytics to support immediate and informed healthcare decisions.

Real-Time Data Integration—The seamless combination of geolocation, patient data, and analytics to provide dynamic and actionable insights.

Real-Time Location Services (RTLS)—Systems that track and monitor the movement of individuals or assets within a defined area in real-time, enhancing operational efficiency and patient safety.

Reinforcement Learning—A type of machine learning where algorithms learn optimal strategies through trial and error, adapting decisions based on real-time feedback.

Remote Patient Monitoring (RPM)—The use of technology to monitor patients' health conditions outside traditional clinical settings, often through wearable devices or IoT-enabled systems.

Sepsis Detection—The identification of sepsis risks through predictive analytics and real-time monitoring of patient health metrics.

Sequential Pattern Mining—The process of identifying patterns in sequential data, useful for understanding temporal relationships.

Smart Contracts—Self-executing contracts with predefined rules encoded on a blockchain, enabling automated and secure data sharing agreements among authorized entities.

Smart Wearables—Devices such as rings, shirts, belts, glasses, and contacts equipped with sensors to monitor health metrics and geolocation data.

Software as a Service (SaaS)—A cloud-based service model that provides software applications over the internet, enabling scalable and efficient healthcare data management.

Streaming Clustering Algorithms—Techniques for dynamically grouping data points in real-time as new information becomes available.

Streaming Data Analytics—The real-time processing and analysis of continuously generated data streams to extract meaningful insights and support dynamic decision-making.

Technology Stacking—The integration of multiple technologies to optimize data processing, storage, and analysis for healthcare applications.

Telehealth Platforms—Systems that enable remote healthcare delivery, including virtual consultations and remote patient monitoring.

Telemedicine—The delivery of healthcare services remotely through telecommunications technology, enabling virtual consultations and remote patient care.

Time-Series Analysis—The examination of data points collected over time to identify trends, seasonality, and patterns.

Virtual Boundaries—Predefined geographic zones established using geofencing technology to monitor and control movements within healthcare settings.

Virtual Reality (VR)—An immersive technology that creates a simulated environment for applications such as medical training, rehabilitation, and patient engagement.

Word Embeddings—Techniques such as Word2Vec and GloVe used in NLP to represent words as vectors, capturing semantic relationships for text analysis.

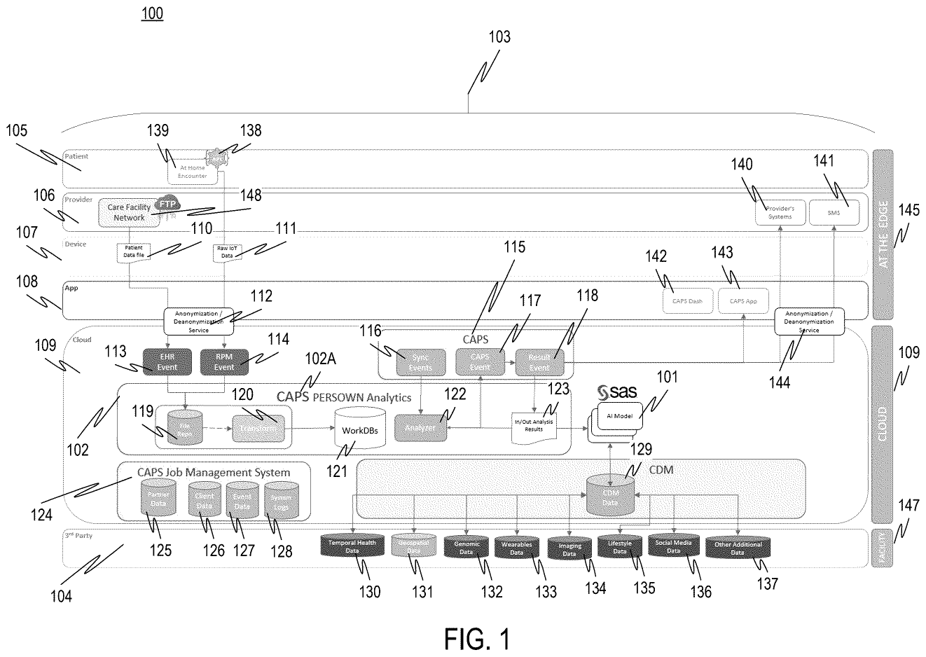

Referring now to FIG. 1, a schematic diagram illustrates an automated platform 100 according to some implementations of the present invention with a Software as a Service AI Model and a controller 102. A non-limiting example of a commonly used software tool sets to analyze data are those tools SAS Institute a software company located in Cary, North Carolina. One or both of the SAS AI Model 101 and the controller 102, may receive data from data sources 103, such as, by way of non-limiting example, one or more of: a patient, a provider, a device, and an app.

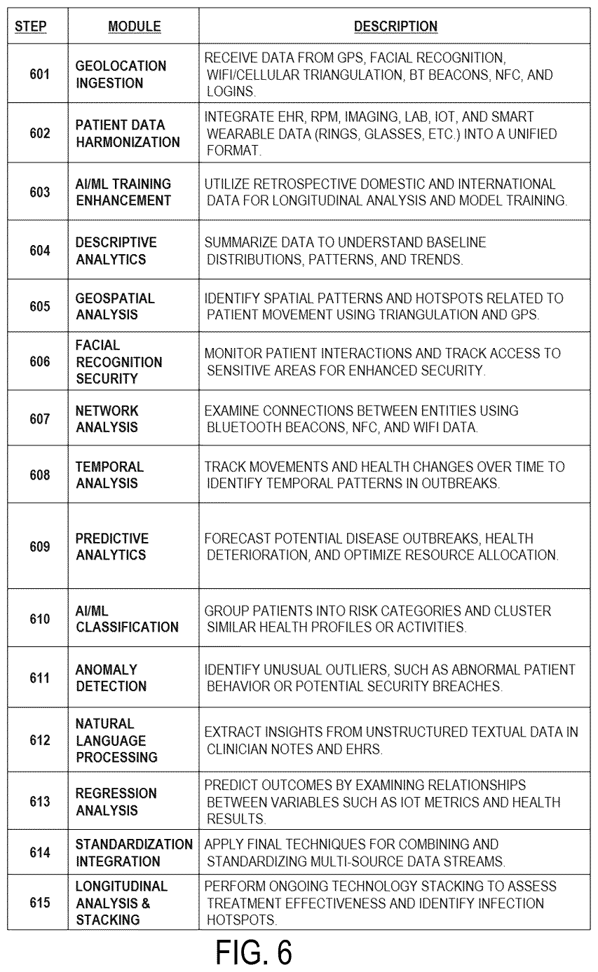

An Automated Platform for processing health records 100 may include one or both of: a Statistical Analysis System (or SAS) AI Model 100, and a Statistical Analysis System (or SAS) AI Model 101. A Controller 102 may include a processor and memory storing executable software executable upon command. The software may be organized in modules, such as those discussed in FIGS. 6 through 16. CAPS Analytics 102A may include, one or more of: File Repo 119, Transform 120, WorkDBs 121 analyzers 122, and In/Out results 123.

Data Sources may include third parties 104, Patients 105, Providers 106, Devices 107, and/or Apps 108, data from devices 107 may include, for example a patient data file 110, and Raw IoT data 111 may be input into a controller 102 for example, via an Application Program Interface 138 or data transfer.

Storage and communication of data may be accomplished via a distributed data communication network, such as the Internet, or a Private Network, accessing a data Cloud 109, the Cloud may include executable software code executable upon command by a controller 102 to perform actions, such as, for example, file repo 119 and data transformation 120 module to supply data to a working database 121, an analyzer 122, and in/Out Analysis Results 123.

In some embodiments, an Anonymization and/or Deanonymization Service 112 may specifically include or exclude personally identifiable information from a Patient Data File 110.

An Electronic Health Record (EHR) Event 113 and/or a Remote Patient Monitoring (RPM) Event 114 may also be stored on a storage device accessible via the Cloud 109.

A Continuous Analytics Processing System (CAPS) may include one or more of: synchronization events 116, CAPS events 117, and result event 118.

A CAPS Job Management System may include, for example, controllers and digital memory storing one or more of: partner data 125, client data 126, event data 127, and system logs 128.

CDM Data may include one or more of: Temporal Health Data 130, Geospatial Data 131, Genomic Data 132, Wearables Data 133, Imaging Data 134, Lifestyle Data 135, Social Media Data 136, and other additional data 137.

Patient supplied data may include data generated from one or more At Home Encounters 139, and may be transceived via an application programing interface 138.

A Provider's System 106 may include a care facility network 148 that may interface via file transfer protocol and generally interface with provider's systems 140 and a SMS module 141.

An App 108 operable on a smart device and include Continuous Analytics Processing System CAPS Dashboard 142 and a CAPS App 143. An Anonymization/Deanonymization Service may also be accessible via the Cloud 109. Controllers and data storage may reside at the Edge 145 and/or a processing 147.

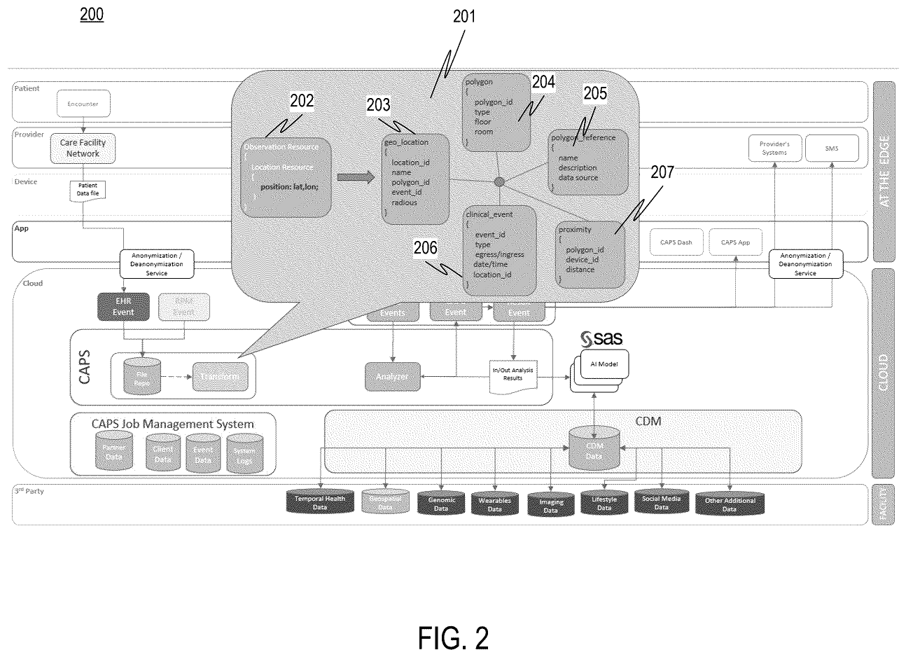

Referring now to FIG. 2, an exemplary automated platform 200 according to some implementations of the present invention may include the Continuous Analytics Processing System (CAPS) aspects, such as those expounded upon and provided in more detail, including, by way of non-limiting example, one or more of: observation resource data; location resource data, and position data which may be coordinate data such as latitude and longitude data; geolocation data; polygons, clinical vents, and proximity information. An exemplary CAPS platform 200 may include a transform 201, with observation resource 202, geolocation 203, polygons 204, polygon references, clinical events 206 and proximity 207.

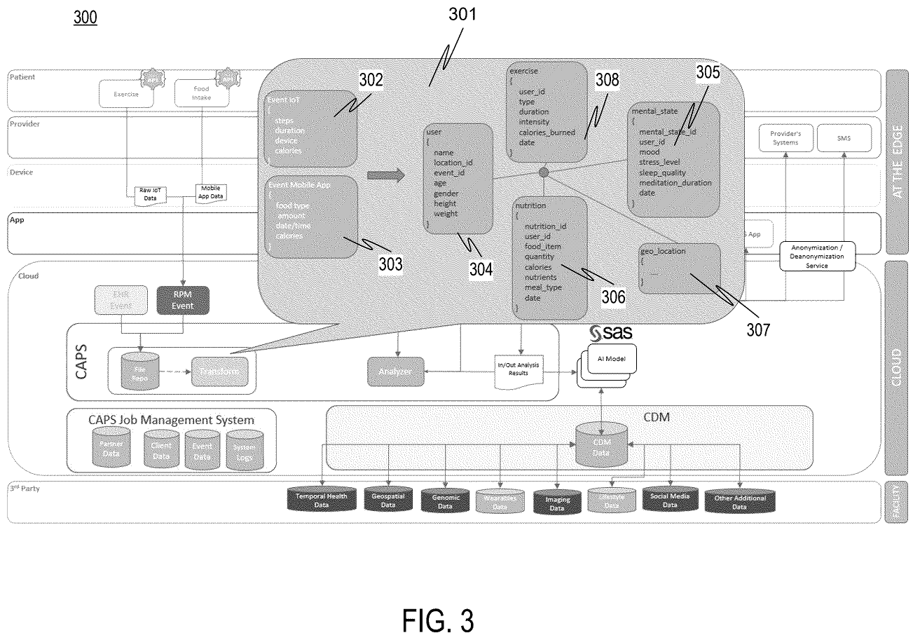

Referring now to FIG. 3 illustrates a schematic diagram of an exemplary automated platform according to some embodiments of the present invention with Transform aspects expounded upon. Transform aspects including, by way of non-limiting example, one or more of: event IoT data, event mobile app data, users; exercise data; nutrition data; mental state data; and geolocation data.. In other embodiments, an exemplary CAPS platform 300, may include a transform 301 including event IoT devices 302, an event mobile app 303, user descriptive data 304, mental state quantifiables 305, nutrition quantifiables 306, and geolocation quantifiables 307. Event IoT 302, may include, for example one or more of: steps, duration, device, calories, etc.

Event IoT 302, may include, for example one or more of: steps, duration, device, calories, etc. Event Mobile App 303 may include, for example one or more of: food type, amount, date/time, calories). User descriptive data 304 may include, for example one or more of: name, location_id, event_id, age, gender, height, weight. Mental State quantifiables may include for example one or more of: mental_state_id, user_id, mood, stress_level, sleep_quality, meditation_duration, date, etc.). Nutrition quantifiables may include, for example one or more of: nutrition_id, user_id, food_item, quantity, calories, nutrients, meal_type, date. Geolocation quantifiables may include, for example one or more of: longitude, latitude, altitude, or other quantity useful for determining a location. Exercise quantifiables may include, for example one or more of: user_id, type, duration, intensity, calories_burned, date, etc.

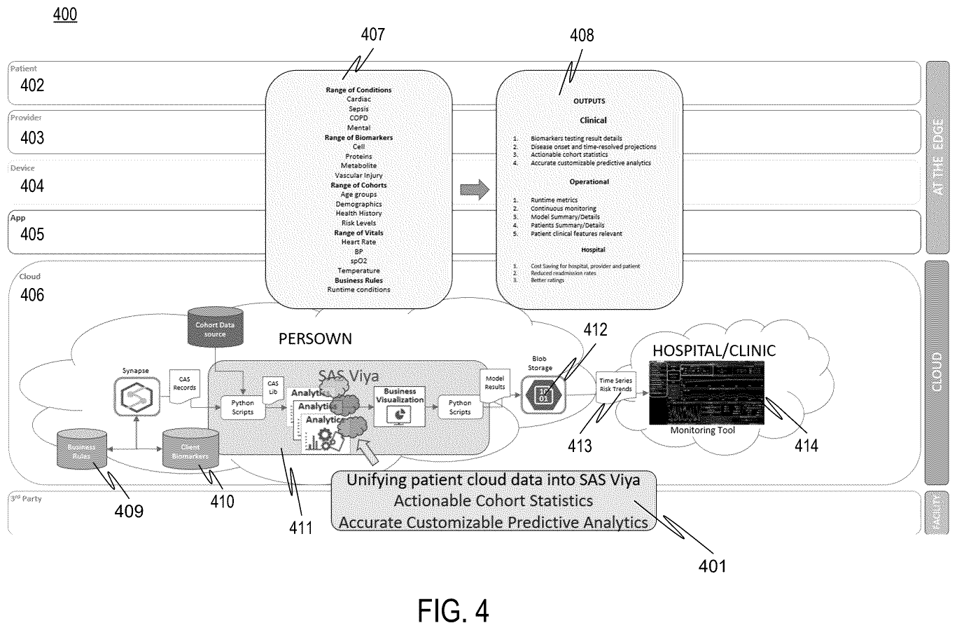

Referring now to FIG. 4, illustrates a schematic diagram of an exemplary automated controllers according to some embodiments of the present invention with SAS Viya actionable cohort statistics to provide accurate customizable predictive analytics. Exemplary Automated Controllers 400 may include, for example processors in logical communication with digital storage storing executable software executable upon command to perform the method steps described herein. Patient data may be unified into SAS Viya Actionable Cohort Statistics Accurate Customizable Predictive Analytics 401. Data may be provided from one or more of: patient 402, health care provider 403, Iot Device 404, an App 405, and Cloud components 406. Ranges 407 of relevant data may include, by way of nonlimiting example, one or more of: Range of Conditions, Range of Biomarkers, Range of Cohorts, Range of Vitals, Business Rules. Outputs 408 of relevant data may include, by way of nonlimiting example, one or more of: Clinical, and Operational, Hospital.

Automated Controllers 400 may also include, for example processors in logical communication with digital storage storing executable software executable upon command to perform process Business rules 409, Client Biomarkers 410, SAS Viya 411, Blob Storage 412, Time Series Risk Trends 413, and Hospital/Clinic Monitoring Tool 414.

A clinical decision support platform enhances healthcare decision-making by providing evidence-based insights, treatment recommendations, and patient-specific guidelines with automated reports and alerts to be generated from the harmonized data from data sources described herein. Coupled with geolocation, the clinical support platform enables such recommendations to be contextualized to the patient's geographical location, considering factors such as local healthcare resources and environmental conditions.

In some embodiments, a security device may be used to actively secure the integrity of data processed, such as the harmonized patient data. Data may be stored within encrypted relational or document-oriented databases that implement encryption at rest and in transit, hardware security module (HSM)-backed key management, and role-based or attribute-based access controls with fine-grained auditing. Trusted execution environments (TEEs), such as secure enclaves, can be used to process and persist sensitive records inside hardware-isolated memory, with remote attestation to verify enclave integrity and HSM-controlled key material preventing unauthorized access even by privileged system software. Tamper-evident append-only storage can be employed through hash-chained journaling of write-once records and periodic external timestamping or notarization to provide integrity and non-repudiation guarantees without reliance on a decentralized ledger. Othe embodiments include a decentralized ledger, such as a blockchain ledger to provide data integrity.

In other embodiments, immutable write-once, read-many (WORM) storage tiers are utilized to preserve audit trails and historical patient records under enforced retention periods, thereby preventing modification or deletion while retaining accessibility for authorized review. A secure data vault can separate direct identifiers from clinical content through tokenization and pseudonymization, maintaining linkage keys within a hardened repository and enforcing controlled re-identification workflows under audited access policies. Policy-driven access can be implemented using attribute-based access control (ABAC) enforced by dedicated policy enforcement points, enabling contextual and purpose-based authorization with comprehensive logging and break-glass mechanisms for emergencies.

Additional embodiments can employ threshold cryptography or secret sharing to shard encryption keys or sensitive data across multiple custodians, mitigating single-point compromise risks, while Merkle tree-based integrity verification is periodically applied over conventional storage snapshots to detect tampering efficiently and to validate dataset completeness. A federated data architecture may keep patient records within institutional repositories governed by a centralized or distributed consent registry that records provenance, consent directives, and cryptographic receipts for each access event. Event-sourced secure storage can maintain patient state as an immutable stream of signed events, with state reconstructions validated against append-only logs and segregated archival of historical snapshots for forensic review.

Further embodiments enable analytics over encrypted data by incorporating homomorphic or secure computation layers that allow limited query operations while data remains encrypted, with keys safeguarded by HSMs and subject to strict governance. The storage and processing environment can be provisioned within HIPAA-compliant virtual private cloud infrastructures featuring isolated networks, private service endpoints, intrusion detection and prevention systems, database activity monitoring, continuous compliance posture management, and automated configuration baselines to maintain confidentiality, integrity, availability, and auditability of the harmonized patient data. These alternatives can be combined or selected according to deployment requirements, enabling a secure storage subsystem that preserves data integrity and provenance while supporting regulated access to clinical information.

Advanced data analytics further amplifies the impact by extracting meaningful patterns and trends from the amalgamated data. This analytical capability allows healthcare providers to identify geographical clusters of specific chronic conditions, assess the effectiveness of interventions, and predict potential outbreaks or disease exacerbations. The geographical insights derived from geolocation data, combined with clinical decision support, and advanced analytics, empower healthcare professionals to formulate personalized care plans that account for the unique challenges posed by each patient's location.

In chronic disease management, proactive interventions are pivotal, and geolocation-based strategies precisely facilitate that. For instance, identifying areas with high prevalence rates of a particular chronic condition allows for targeted public health campaigns and resource allocation. Additionally, geolocation data can be leveraged to monitor patient adherence to treatment plans, track lifestyle changes, and offer timely interventions when deviations occur. This comprehensive approach fosters a patient-centric model that not only manages chronic diseases more effectively but also promotes preventative measures, improving overall health outcomes and reducing the burden on healthcare systems. Ultimately, the integration of geolocation, clinical decision support, and advanced data analytics marks a paradigm shift towards precision and context-aware chronic disease management.

The integration of geolocation with a clinical decision support platform and advanced data analytics offers a groundbreaking paradigm for managing cardiovascular diseases, encompassing conditions such as coronary artery disease, heart failure, arrhythmias, and hypertension. Geolocation data adds a spatial dimension to cardiovascular care, providing insights into environmental factors, lifestyle patterns, and proximity to healthcare resources. This information, when harnessed by a clinical decision support platform, enables healthcare professionals to make more informed decisions tailored to the patient's geographical context. In the case of coronary artery disease, geolocation can assist in identifying areas with a higher prevalence of risk factors such as air pollution or limited access to healthy food options.

A clinical decision support platform, enriched with geospatial data, can recommend targeted lifestyle modifications and preventive measures based on the patient's location. Advanced data analytics contribute by identifying spatial patterns in cardiovascular events, enabling healthcare providers to implement proactive interventions and optimize emergency response strategies. For heart failure patients, geolocation can aid in monitoring fluid retention and physical activity levels. When integrated with clinical decision support, this data can prompt timely adjustments to medication regimens or lifestyle recommendations. Advanced analytics facilitate the prediction of exacerbations by analyzing historical geospatial trends, allowing for anticipatory interventions and hospital resource optimization. In the realm of arrhythmias, geolocation combined with clinical decision support enhances the management of patients with implantable devices.

Real-time location data can assist in remote monitoring, ensuring prompt detection of irregular heart rhythms. The platform can provide decision support for adjusting device parameters or initiating interventions based on geospatial context. Advanced analytics contribute by analyzing geolocation trends to identify potential triggers for arrhythmic events. In hypertension management, geolocation offers insights into environmental stressors and local healthcare accessibility.

A clinical decision support platform can leverage this information to tailor medication regimens and lifestyle recommendations. Advanced data analytics assist in identifying geographical trends in hypertension prevalence and response to interventions. Collectively, the integration of geolocation, clinical decision support, and advanced analytics in cardiovascular disease management brings a holistic and patient-centered approach. This innovative combination not only improves individual patient care but also empowers healthcare systems to implement targeted public health initiatives, leading to more effective prevention and management of cardiovascular diseases on a broader scale.

Integration of geolocation with a clinical decision support platform and advanced data analytics holds significant promise in transforming the management of nephrological conditions, particularly chronic kidney disease (CKD). Geolocation data adds a spatial context to CKD care, offering insights into environmental factors, socioeconomic conditions, and access to healthcare resources. This information, when combined with a clinical decision support platform, empowers nephrologists to make informed decisions that are tailored to the patient's geographical context.

For patients with CKD, geolocation can play a pivotal role in assessing lifestyle factors, including dietary habits, exposure to environmental toxins, and proximity to healthcare facilities.

A clinical decision support platform, enriched with geospatial data, can provide personalized treatment plans that consider these factors. Advanced data analytics further contribute by identifying spatial patterns related to CKD prevalence and risk factors, enabling proactive public health measures and targeted interventions. In the context of renal replacement therapy, geolocation can assist in optimizing access to dialysis centers and transplantation facilities. This information, integrated into a clinical decision support system, facilitates real-time decision-making regarding treatment modalities, transplant suitability, and resource allocation. Advanced analytics enhance this by analyzing geospatial trends to predict patient needs, optimize transportation logistics, and ensure timely interventions. For remote patient monitoring in CKD, geolocation data can aid in tracking patients' adherence to treatment plans and identifying potential barriers to care. The clinical decision support platform, when leveraging this information, can prompt timely adjustments to medication regimens or lifestyle recommendations.

Advanced analytics contribute by analyzing geolocation trends to predict disease progression and optimize the allocation of telehealth resources. In the realm of CKD prevention, geolocation combined with clinical decision support can facilitate targeted public health campaigns in areas with a higher prevalence of risk factors. By identifying geographical clusters of CKD incidence, healthcare providers can implement community-based interventions and educational programs. Advanced data analytics assist in assessing the effectiveness of these interventions by analyzing geospatial trends in disease prevalence over time. In conclusion, the integration of geolocation, clinical decision support, and advanced analytics in nephrological care, particularly for CKD, represents a transformative approach. This comprehensive strategy not only enhances individual patient care but also contributes to proactive public health initiatives, ultimately improving outcomes for patients with nephrological conditions on a broader scale.

The integration of geolocation with a clinical decision support platform and advanced data analytics presents a multifaceted approach to addressing post-surgical complications, sepsis, and hospital readmissions. Geolocation data adds a helpful spatial context to post-surgical care, offering insights into the patient's environment, proximity to healthcare facilities, and potential sources of infection. When incorporated into a clinical decision support platform, this geospatial information enables healthcare professionals to make data-driven decisions tailored to the patient's geographical context, optimizing post-surgical recovery.

In cases involving post-surgical complications, geolocation data can assist in monitoring patients' recovery trajectories and adherence to postoperative care plans. The clinical decision support platform, enriched with geospatial insights, facilitates personalized interventions based on the patient's environment and accessibility to follow-up care. Advanced data analytics contribute by identifying spatial patterns related to post-surgical complications, allowing for targeted preventive measures and optimized resource allocation in high-risk areas. For sepsis management, geolocation can play a helpful role in early detection and intervention. Real-time tracking of patients' geolocation data can help identify potential sources of infection, track the progression of symptoms, and prompt timely clinical interventions.

The clinical decision support platform, integrated with geospatial information, assists healthcare providers in tailoring sepsis treatment plans based on the patient's location and potential exposure risks. Advanced analytics contribute by analyzing geolocation trends to predict sepsis outbreaks and optimize response strategies. In the context of hospital readmissions, geolocation data helps to provide understanding of the patient's post-discharge environment and potential challenges. The clinical decision support platform utilizes this information to customize post-discharge care plans, ensuring that they align with the patient's geographical context and access to follow-up care. Advanced data analytics contribute by analyzing geospatial trends related to readmission rates, facilitating targeted interventions, and improving the effectiveness of postoperative care strategies. Collectively, the integration of geolocation, clinical decision support, and advanced analytics in post-surgical complications, sepsis, and hospital readmissions offers a comprehensive and patient-centered approach. This innovative combination not only improves individual patient outcomes but also empowers healthcare systems to implement proactive measures, enhance postoperative care, and reduce the burden of complications and readmissions on healthcare resources.

Methods and processes utilizing a clinical decision support system according to the present invention, identify and prevent/treat infectious diseases represents a comprehensive approach to transforming healthcare practices. Geolocation data plays a pivotal role in this method, utilizing technologies such as GPS trackers, Wi-Fi triangulation, and cellular triangulation to collect and analyze location-specific information related to infections caused by pathogens. This real-time geolocation data becomes the foundation for a multi-faceted strategy. Pathogen identification is a crucial step in the process, where specific pathogens causing infections are identified based on the geolocation data collected. This molecular diagnostic approach ensures accurate and targeted responses to infections. Concurrently, the method involves analyzing antibiotic use patterns across different locations using advanced drug use analysis means. This data-driven analysis forms the basis for providing personalized and targeted antibiotic prescriptions to healthcare providers through prescription recommendation means.

Methods of the present invention include an antibiotic stewardship component, generating real-time insights into antibiotic use and recommending interventions to promote appropriate prescribing practices. This could involve identifying areas with high rates of unnecessary antibiotic use and implementing educational programs or decision support tools for healthcare providers. The geolocation data is also helpful in tracking the real-time spread of specific pathogens, enabling rapid identification and containment of outbreaks through outbreak prevention and control means. To address the emerging threat of antimicrobial resistance (AMR), the method incorporates AMR surveillance and management means. By linking antibiotic use data with pathogen resistance patterns, valuable insights are gained into the emergence and spread of AMR, guiding public health interventions and the development of new antibiotics. The decision support tools for healthcare providers utilize real-time insights for informed decision-making in antibiotic prescriptions.

Additionally, educational programs are implemented to address areas with high rates of unnecessary antibiotic use, promoting optimized antibiotic stewardship. The method is dynamic and responsive, incorporating a system of sending alerts to healthcare providers based on real-time insights, personalized prescription recommendations, and educational materials, ensuring timely and effective responses to infectious diseases. This comprehensive approach represents a paradigm shift in infectious disease management, leveraging advanced technologies and data analytics for improved patient outcomes and public health.

As shown in FIG. 6, some embodiments of the present invention include modules directed to infection control utilizing patient tracing/tracking and monitoring technology, a controller, such as a cloud-based server, may implement one or more of the following method steps:
At Step 601, receiving geolocation data from a plurality of sources, including GPS trackers, facial recognition, Wi-Fi triangulation, cellular triangulation, Bluetooth beacons, near-field communication, login credentials, and check-ins;
At Step 602, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 603, utilizing retrospective Domestic and International data to enhance AI/ML training and longitudinal data analysis;
At Step 604, employing descriptive analytics to summarize and understand the distribution, patterns, and trends in the collected data;
At Step 605, applying geospatial analysis to identify spatial patterns, hotspots, and trends related to patient movement and location using GPS, Wi-Fi triangulation, and cellular triangulation data;
At Step 606, implementing facial recognition algorithms to enhance security, monitor patient interactions, and track access to sensitive areas;
At Step 607, conducting network analysis to examine relationships and connections between entities in a network, applicable to Bluetooth beacons, NFC, and Wi-Fi triangulation;
At Step 608, performing temporal analysis to track patient movements, identify temporal patterns in infection outbreaks, and monitor changes in health data over time;
At Step 609, using predictive analytics to forecast potential disease outbreaks, predict patient health deterioration, and optimize resource allocation;
At Step 610, applying (AI/ML) classification and clustering to classify patients into risk groups, clustering similar patient profiles, and categorizing different types of healthcare activities;
At Step 611, employing anomaly detection to identify unusual patterns or outliers in the data, such as abnormal patient behavior, potential security breaches, or anomalies in health metrics;
At Step 612, leveraging natural language processing (NLP) to extract insights from textual data in EHRs, clinician notes, and patient communications;
At Step 613, utilizing regression analysis to examine the relationship between variables and predict outcomes, such as predicting patient health outcomes based on various factors, including IoT data and wearable device metrics;
At Step 614, integrating and harmonizing techniques for combining and standardizing data from various sources; and
At Step 615, performing longitudinal data analysis to understand patient health trends, assess effectiveness of treatments over time, and support longitudinal studies, wherein, the method further involves continuous technology stacking for analyzing the harmonized data to identify patterns indicative of potential infection hotspots and implementing infection control measures based on the analysis of geolocation data, thereby contributing to the prevention and containment of infectious diseases.

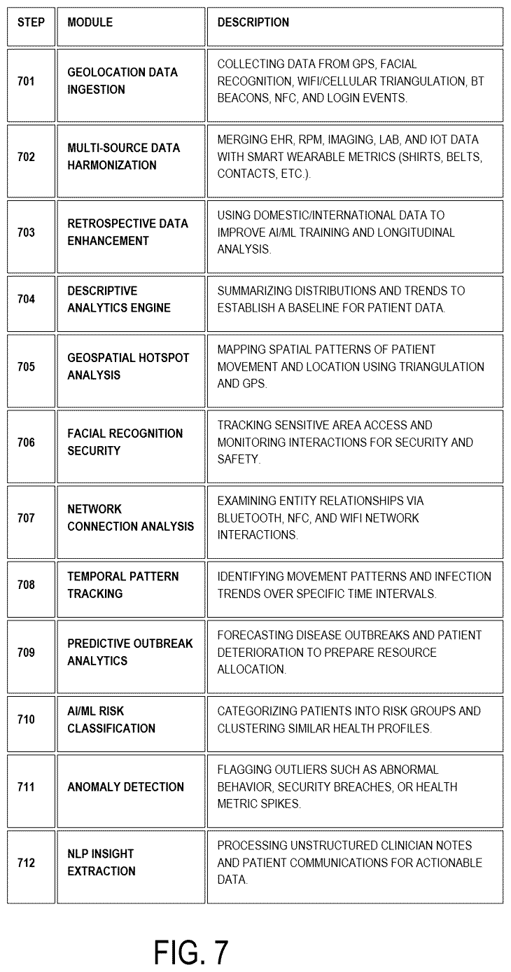

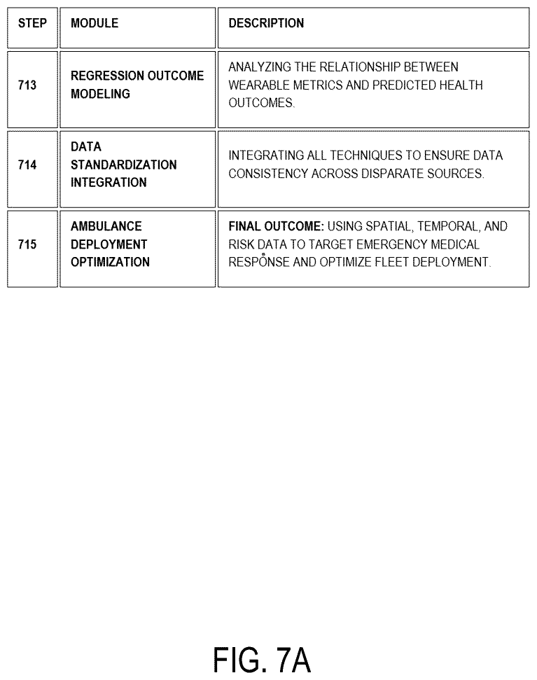

As shown in FIGS. 7-7A, some embodiments of the present invention include modules directed to optimizing ambulance deployment based on geolocation data, a controller, such as a cloud-based server, may implement one or more of the following method steps:
At Step 701, receiving geolocation data from a plurality of sources, including GPS trackers, facial recognition, Wi-Fi triangulation, cellular triangulation, Bluetooth beacons, near-field communication, login credentials, and check-ins;
At Step 702, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 703, utilizing retrospective Domestic and International data to enhance AI/ML training and longitudinal data analysis;
At Step 704, employing descriptive analytics to summarize and understand the distribution, patterns, and trends in the collected data;
At Step 705, applying geospatial analysis to identify spatial patterns, hotspots, and trends related to patient movement and location using GPS, Wi-Fi triangulation, and cellular triangulation data;
At Step 706, implementing facial recognition algorithms to enhance security, monitor patient interactions, and track access to sensitive areas;
At Step 707, conducting network analysis to examine relationships and connections between entities in a network, applicable to Bluetooth beacons, NFC, and Wi-Fi triangulation;
At Step 708, performing temporal analysis to track patient movements, identify temporal patterns in infection outbreaks, and monitor changes in health data over time;
At Step 709, using predictive analytics to forecast potential disease outbreaks, predict patient health deterioration, and optimize resource allocation;
At Step 710, applying (AI/ML) classification and clustering to classify patients into risk groups, clustering similar patient profiles, and categorizing different types of healthcare activities;
At Step 711, employing anomaly detection to identify unusual patterns or outliers in the data, such as abnormal patient behavior, potential security breaches, or anomalies in health metrics;
At Step 712, leveraging natural language processing (NLP) to extract insights from textual data in EHRs, clinician notes, and patient communications;
At Step 713, utilizing regression analysis to examine the relationship between variables and predict outcomes, such as predicting patient health outcomes based on various factors, including IoT data and wearable device metrics;
At Step 714, integrating and harmonizing techniques for combining and standardizing data from various sources; and
At Step 715, performing longitudinal data analysis to understand patient health trends, assessing the effectiveness of treatments over time, and supporting longitudinal studies. In some embodiments, the method may further involve optimizing ambulance deployment based on the analysis of geolocation data, spatial patterns, patient risk classifications, temporal trends, and anomaly detection, thereby contributing to efficient and targeted emergency medical response.

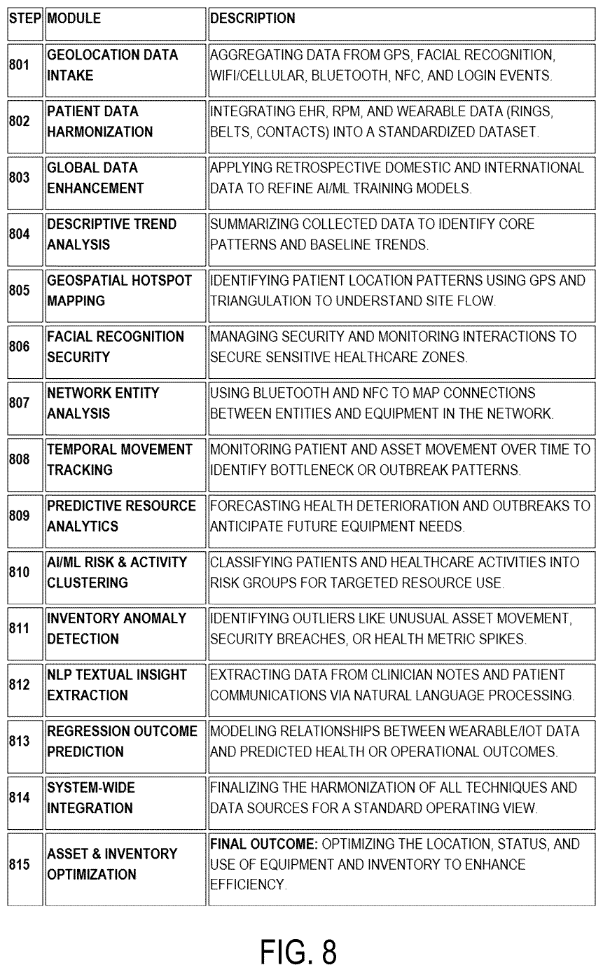

As seen in FIG. 8, some embodiments of the present invention include modules directed to geolocation-based asset and inventory management within healthcare environments, a controller, such as a cloud-based server, may implement one or more of the following method steps:
At Step 801, receiving geolocation data from a plurality of sources, including GPS trackers, facial recognition, Wi-Fi triangulation, cellular triangulation, Bluetooth beacons, near-field communication, login credentials, and check-ins;
At Step 802, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 803, utilizing retrospective Domestic and International data to enhance AI/ML training and longitudinal data analysis;
At Step 804, employing descriptive analytics to summarize and understand the distribution, patterns, and trends in the collected data;
At Step 805, applying geospatial analysis to identify spatial patterns, hotspots, and trends related to patient movement and location using GPS, Wi-Fi triangulation, and cellular triangulation data;
At Step 806, implementing facial recognition algorithms to enhance security, monitor patient interactions, and track access to sensitive areas;
At Step 807, conducting network analysis to examine relationships and connections between entities in a network, applicable to Bluetooth beacons, NFC, and Wi-Fi triangulation;
At Step 808, performing temporal analysis to track patient movements, identify temporal patterns in infection outbreaks, and monitor changes in health data over time;
At Step 809, using predictive analytics to forecast potential disease outbreaks, predict patient health deterioration, and optimize resource allocation;
At Step 810, applying AI/ML classification and clustering to classify patients into risk groups, clustering similar patient profiles, and categorizing different types of healthcare activities;
At Step 811, employing anomaly detection to identify unusual patterns or outliers in the data, such as abnormal patient behavior, potential security breaches, or anomalies in health metrics;
At Step 812, leveraging natural language processing (NLP) to extract insights from textual data in EHRs, clinician notes, and patient communications;
At Step 813, utilizing regression analysis to examine the relationship between variables and predict outcomes, such as predicting patient health outcomes based on various factors, including IoT data and wearable device metrics;
At Step 814, integrating and harmonizing techniques for combining and standardizing data from various sources;
At Step 815, performing longitudinal data analysis to understand patient health trends, assess the effectiveness of treatments over time, and support longitudinal studies,
wherein, the method further involves geolocation-based asset and inventory management by optimizing the location, status, and utilization of healthcare assets, equipment, and inventory based on the analysis of geolocation data, spatial patterns, patient classifications, and anomaly detection, thereby contributing to enhanced efficiency and resource utilization in healthcare environments.

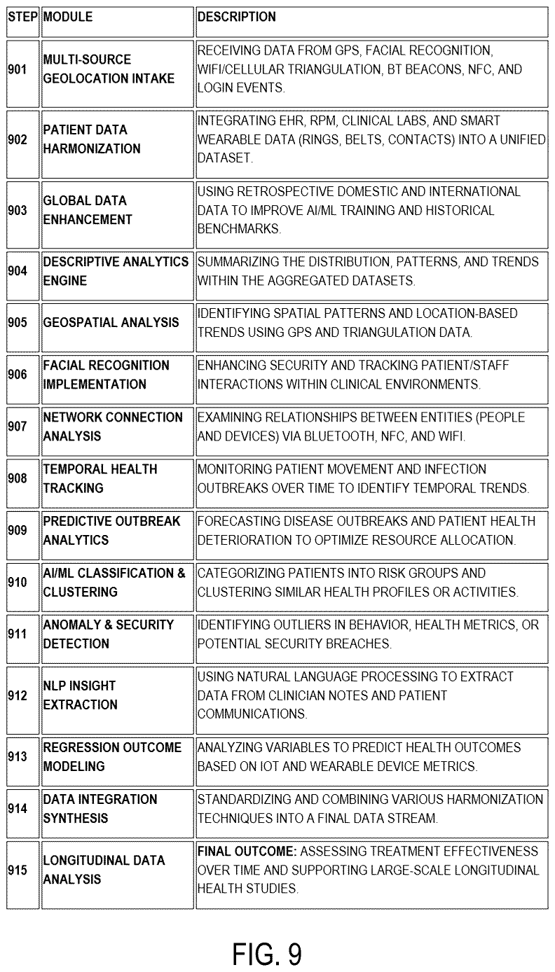

As shown in FIG. 9, some embodiments of the present invention include modules directed to optimizing ambulance deployment based on geolocation data, a controller, such as a cloud-based server, may implement one or more of the following method steps:
At Step 901, receiving geolocation data from a plurality of sources, including GPS trackers, facial recognition, Wi-Fi triangulation, cellular triangulation, Bluetooth beacons, near-field communication, login credentials, and check-ins;
At Step 902, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 903, utilizing retrospective Domestic and International data to enhance AI/ML training and longitudinal data analysis;
At Step 904, employing descriptive analytics to summarize and understand the distribution, patterns, and trends in the collected data;
At Step 905, applying geospatial analysis to identify spatial patterns, hotspots, and trends related to patient movement and location using GPS, Wi-Fi triangulation, and cellular triangulation data;
At Step 906, implementing facial recognition algorithms to enhance security, monitor patient interactions, and track access to sensitive areas;
At Step 907, conducting network analysis to examine relationships and connections between entities in a network, applicable to Bluetooth beacons, NFC, and Wi-Fi triangulation;
At Step 908, performing temporal analysis to track patient movements, identify temporal patterns in infection outbreaks, and monitor changes in health data over time;
At Step 909, using predictive analytics to forecast potential disease outbreaks, predict patient health deterioration, and optimize resource allocation;
At Step 910, applying AI/ML classification and clustering to classify patients into risk groups, clustering similar patient profiles, and categorizing different types of healthcare activities;
At Step 911, employing anomaly detection to identify unusual patterns or outliers in the data, such as abnormal patient behavior, potential security breaches, or anomalies in health metrics;
At Step 912, leveraging natural language processing (NLP) to extract insights from textual data in EHRs, clinician notes, and patient communications;
At Step 913, utilizing regression analysis to examine the relationship between variables and predict outcomes, such as predicting patient health outcomes based on various factors, including IoT data and wearable device metrics;
At Step 914, integrating and harmonizing techniques for combining and standardizing data from various sources; and
At Step 915, performing longitudinal data analysis to understand patient health trends, assess the effectiveness of treatments over time, and support longitudinal studies.

In some embodiments, the present invention may include common programming libraries and tools to enable geolocation-based geofencing to establish virtual boundaries and monitor patient movements within defined areas, triggering alerts, and optimizing healthcare activities based on the analysis of geolocation data, spatial patterns, patient classifications, and anomaly detection, thereby contributing to enhanced patient safety and healthcare efficiency within designated zones.

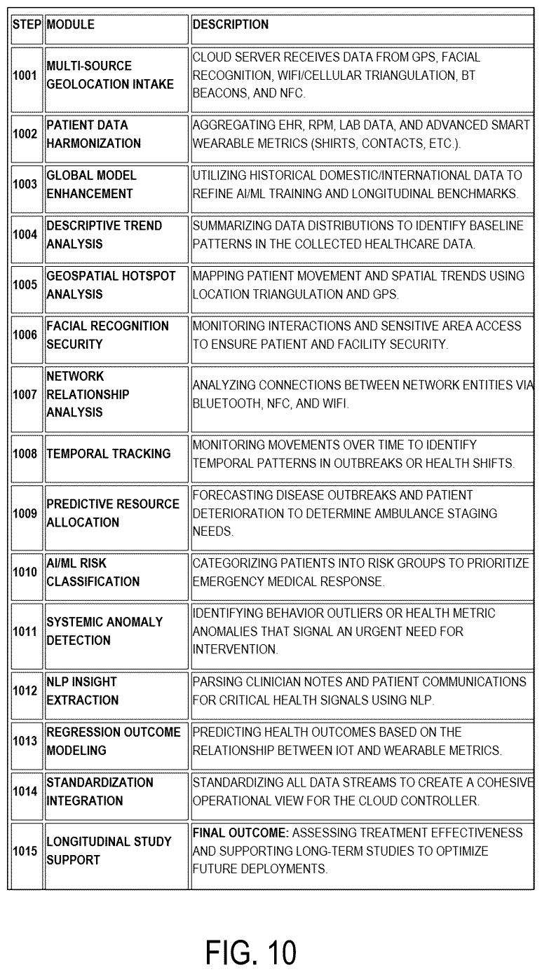

As shown in FIG. 10, some embodiments of the present invention include modules directed to optimizing ambulance deployment based on geolocation data, a controller, such as a cloud-based server, may implement one or more of the following method steps:
At Step 1001, receiving geolocation data from a plurality of sources, including GPS trackers, facial recognition, Wi-Fi triangulation, cellular triangulation, Bluetooth beacons, near-field communication, login credentials, and check-ins;
At Step 1002, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 1003, utilizing retrospective Domestic and International data to enhance AI/ML training and longitudinal data analysis;
At Step 1004, employing descriptive analytics to summarize and understand the distribution, patterns, and trends in the collected data;
At Step 1005, applying geospatial analysis to identify spatial patterns, hotspots, and trends related to patient movement and location using GPS, Wi-Fi triangulation, and cellular triangulation data;
At Step 1006, implementing facial recognition algorithms to enhance security, monitor patient interactions, and track access to sensitive areas;
At Step 1007, conducting network analysis to examine relationships and connections between entities in a network, applicable to Bluetooth beacons, NFC, and Wi-Fi triangulation;
At Step 1008, performing temporal analysis to track patient movements, identify temporal patterns in infection outbreaks, and monitor changes in health data over time;
At Step 1009, using predictive analytics to forecast potential disease outbreaks, predict patient health deterioration, and optimize resource allocation;
At Step 1010, applying AI/ML classification and clustering to classify patients into risk groups, clustering similar patient profiles, and categorizing different types of healthcare activities;
At Step 1011, employing anomaly detection to identify unusual patterns or outliers in the data, such as abnormal patient behavior, potential security breaches, or anomalies in health metrics;
At Step 1012, leveraging natural language processing (NLP) to extract insights from textual data in EHRs, clinician notes, and patient communications;
At Step 1013, utilizing regression analysis to examine the relationship between variables and predict outcomes, such as predicting patient health outcomes based on various factors, including IoT data and wearable device metrics;
At Step 1014, integrating and harmonizing techniques for combining and standardizing data from various sources; and
At Step 1015, performing longitudinal data analysis to understand patient health trends, assessing the effectiveness of treatments over time, and supporting longitudinal studies.

In some embodiments of the present invention, the controller may be operative to perform remote patient monitoring and intervention based on the analysis of geolocation data, spatial patterns, patient classifications, anomaly detection, and predictive analytics, thereby facilitating timely and personalized healthcare interventions for remote patients.

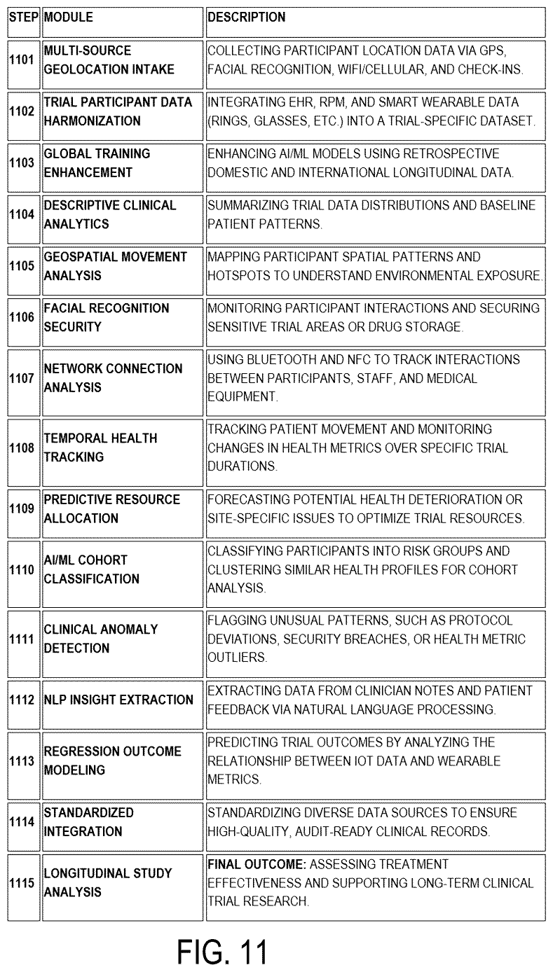

As shown in FIG. 11, in some embodiments of the present invention directed to geolocation-based clinical trial research and data management, a controller, such as a cloud-based server, may implement one or more of the following method steps:
At Step 1101, receiving geolocation data from a plurality of sources, including GPS trackers, facial recognition, Wi-Fi triangulation, cellular triangulation, Bluetooth beacons, near-field communication, login credentials, and check-ins;
At Step 1102, harmonizing patient data from various sources, comprising Electronic Health Record (EHR) data from acute care site(s), Remote Patient Monitoring data, Physician offices data, Imaging data, Nursing home data, Home care data, Clinical lab data, Point-of-care device data, Internet of Things (IoT) data, and data from smart wearables including rings, shirts, belts, glasses, contacts, and similar devices;
At Step 1103, utilizing retrospective Domestic and International data to enhance AI/ML training and longitudinal data analysis;
At Step 1104, employing descriptive analytics to summarize and understand the distribution, patterns, and trends in the collected data;
At Step 1105, applying geospatial analysis to identify spatial patterns, hotspots, and trends related to patient movement and location using GPS, Wi-Fi triangulation, and cellular triangulation data;
At Step 1106, implementing facial recognition algorithms to enhance security, monitor patient interactions, and track access to sensitive areas;
At Step 1107, conducting network analysis to examine relationships and connections between entities in a network, applicable to Bluetooth beacons, NFC, and Wi-Fi triangulation;
At Step 1108, performing temporal analysis to track patient movements, identify temporal patterns in infection outbreaks, and monitor changes in health data over time;
At Step 1109, using predictive analytics to forecast potential disease outbreaks, predict patient health deterioration, and optimize resource allocation;
At Step 1110, applying AI/ML classification and clustering to classify patients into risk groups, clustering similar patient profiles, and categorizing different types of healthcare activities;
At Step 1111, employing anomaly detection to identify unusual patterns or outliers in the data, such as abnormal patient behavior, potential security breaches, or anomalies in health metrics;
At Step 1112, leveraging natural language processing (NLP) to extract insights from textual data in EHRs, clinician notes, and patient communications;
At Step 1113, utilizing regression analysis to examine the relationship between variables and predict outcomes, such as predicting patient health outcomes based on various factors, including IoT data and wearable device metrics;
At Step 1114, integrating and harmonizing techniques for combining and standardizing data from various sources; and
At Step 1115, performing longitudinal data analysis to understand patient health trends, assess the effectiveness of treatments over time, and support longitudinal studies.

In some embodiments, the controller may be operative to provide geolocation-based clinical trial research and data management by optimizing participant monitoring, safety, and data integrity through the analysis of geolocation data, spatial patterns, patient classifications, anomaly detection, and predictive analytics, thereby contributing to enhanced clinical trial efficiency and efficacy.

Some implementations of the present invention may include a controller operative to incorporate data from wearable devices, including smart rings, shirts, belts, glasses, contacts, or similar devices, to enhance the accuracy of patient location tracking.

Some implementations of the present invention may include retrospective Domestic and International data utilized including historical infection data, population density information, and travel patterns, contributing to a comprehensive analysis for infection control.

Some implementations of the present invention may include a controller operative to generate visualizations and reports to facilitate a clear understanding of distribution, patterns, and trends in the collected data for infection control decision-making.

Some implementations of the present invention may include a controller operative to incorporate real-time data from GPS, Wi-Fi triangulation, and cellular triangulation to dynamically identify and update spatial patterns, hotspots, and trends related to patient movement and location.

Some implementations of the present invention may include a controller operative to evaluate the strength and frequency of connections between entities in the network, aiding in the identification of potential transmission routes and infection spread.

Some implementations of the present invention may include a controller operative to utilize AI/ML models to predict the likelihood of disease outbreaks based on a combination of historical data, patient health metrics, and environmental factors.

AI/ML classification and clustering may employ algorithms to adaptively categorize patients into risk groups, considering evolving patterns and emerging trends in healthcare data.

In some implementations, anomaly detection may use AI/ML techniques to identify abnormal patient behavior, potential security breaches, or anomalies in health metrics with a high degree of accuracy.

In some implementations, natural language processing (NLP) in step (l) is further configured to extract specific information related to infectious disease symptoms, exposure history, and treatment responses from textual data in EHRs, clinician notes, and patient communications.

In some implementations, regression analysis in step (m) considers a wide range of variables, including IoT data and wearable device metrics, to predict patient health outcomes and support personalized treatment plans.

In some implementations, longitudinal data analysis in step (o) involves monitoring patient health trends over an extended period, identifying patterns indicative of chronic conditions, and supporting evidence-based decision-making in infection control measures.

In some implementations, continuous technology stacking in the concluding statement involves integrating emerging technologies and algorithms to enhance the efficiency and accuracy of infection outbreak detection and control measures.

In some implementations, predictive analytics in step (i) is specifically configured for sepsis detection, employing machine learning models trained on a combination of historical patient data, vital signs, and laboratory results to forecast the potential onset of sepsis and optimize resource allocation for timely intervention.

In some implementations, adapting the AI/ML classification and clustering in may include categorizing patients into risk groups specific to cardiovascular diseases, considering relevant health metrics, lifestyle factors, and historical cardiovascular data, thereby facilitating targeted analysis and preventative measures for cardiovascular health within the infection control framework.

In some implementations, cardiovascular diseases categorized in step (j) include but are not limited to coronary artery disease, heart failure, arrhythmias, and hypertension, enabling the identification of distinct infection control strategies tailored to the specific cardiovascular conditions of patients within the risk groups.

In some implementations, adapting the AI/ML classification and clustering in step (j) to categorize patients into risk groups specific to chronic kidney disease, incorporating relevant health metrics, renal function indicators, and historical data related to kidney health, thereby facilitating targeted analysis and infection control measures tailored to individuals with chronic kidney disease.

In some implementations, applying the AI/ML classification and clustering in step (j) to categorize patients into risk groups specifically associated with post-operative readmission, utilizing historical surgical data, post-operative complications, and patient recovery patterns, thereby enabling the identification of infection control measures tailored to reduce the risk of post-operative readmission due to infectious complications.

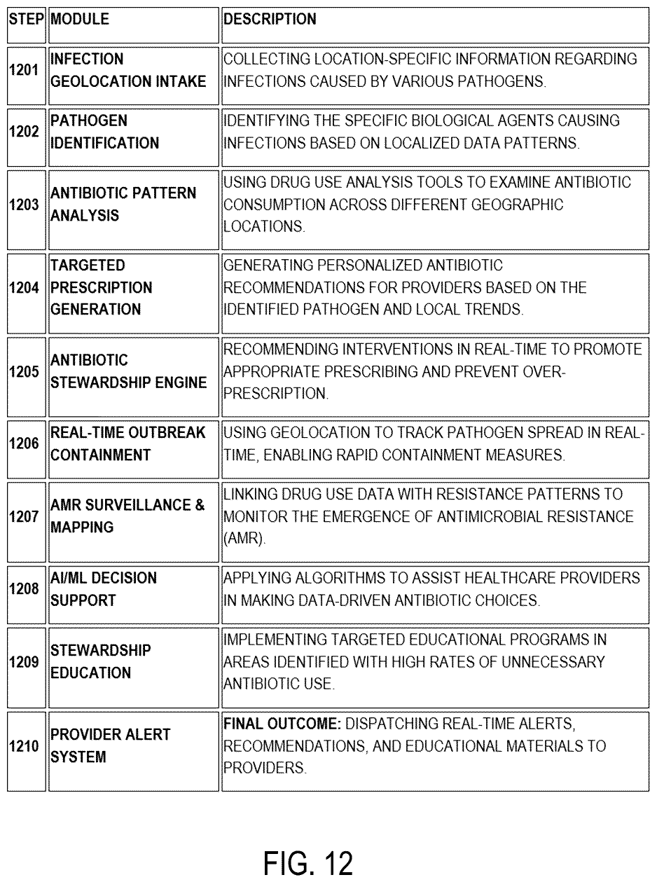

As shown in FIG. 12, some embodiments of the present invention include modules directed to a clinical decision support system to identify and prevent/treat infectious diseases, wherein a controller, such as a cloud-based server, may implement one or more of the following method steps:
At Step 1201, collecting geolocation data to receive and analyze location-specific information related to infections caused by pathogens;
At Step 1202, identifying specific pathogens causing infections based on the collected geolocation data;
At Step 1203, analyzing antibiotic use patterns in different locations using drug use analysis means;
At Step 1204, providing personalized and targeted antibiotic prescriptions to healthcare providers based on the identified pathogens and antibiotic use patterns through prescription recommendation means;
At Step 1205, generating real-time insights into antibiotic use across diverse locations and recommending interventions to promote appropriate antibiotic prescribing practices through antibiotic stewardship means;
At Step 1206, utilizing geolocation data to track the real-time spread of specific pathogens, enabling rapid identification and containment of outbreaks through outbreak prevention and control means;
At Step 1207, linking antibiotic use data with pathogen resistance patterns through antimicrobial resistance (AMR) surveillance and management means, providing insights into the emergence and spread of AMR;
At Step 1208, utilizing AI/ML algorithms and decision support tools for healthcare providers to make informed decisions in antibiotic prescriptions based on real-time insights;
At Step 1209, implementing educational programs for healthcare providers to address areas with high rates of unnecessary antibiotic use, promoting optimized antibiotic stewardship; and
At Step 1210, sending alerts to healthcare providers based on real-time insights, personalized prescription recommendations, and educational materials.

In some implementations, pathogen identification may include molecular diagnostic techniques, genotypic analysis, or other methods for accurate identification of pathogens.

In some implementations, drug use analysis may utilize data analytics algorithms to identify patterns and trends in antibiotic prescriptions across different geographical locations.

In some implementations, antibiotic stewardship may recommend interventions such as educational programs or decision support tools based on the real-time insights into antibiotic use.

In some implementations, outbreak prevention and control may employ geospatial analysis to identify spatial patterns, hotspots, and trends related to the spread of specific pathogens.

In some embodiments of the present invention directed to AMR surveillance and management utilizing AI/ML algorithms to analyze the linkage between antibiotic use data and pathogen resistance patterns and provide actionable insights for public health interventions.

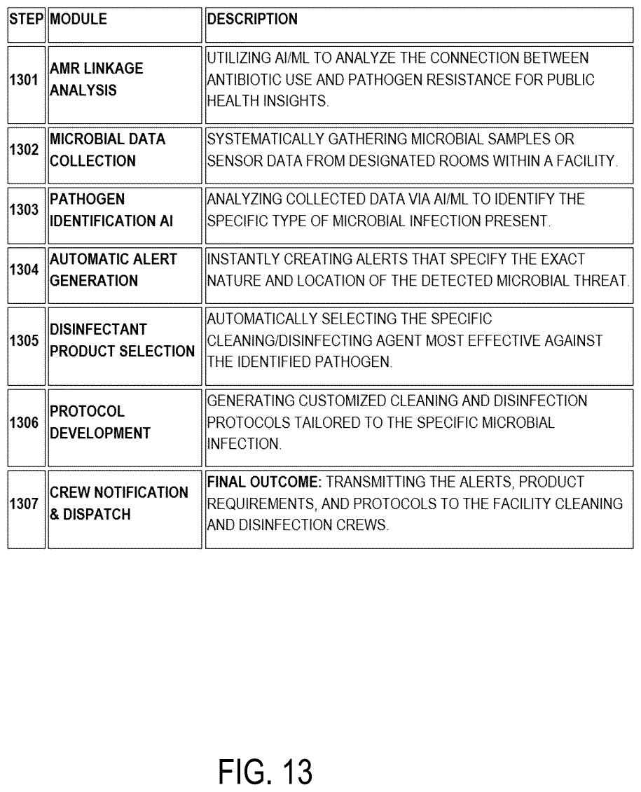

As shown in FIG. 13, a controller, such as a cloud-based server, may implement one or more of the following method steps:
At Step 1301, utilize AI/ML algorithms to analyze the linkage between antibiotic use data and pathogen resistance patterns, providing actionable insights for public health interventions beginning with alerting and guiding facility cleaning and disinfection crews in response to microbial infections, comprising:
At Step 1302, collection of microbial data from designated rooms within a facility;
At Step 1303, analysis of the collected microbial data using an AI/ML algorithm for the identification of specific microbial infections;
At Step 1304, automatic generation of alerts specifying the type of microbial infection detected;
At Step 1305, selection of the appropriate cleaning and disinfecting product tailored to the identified microbial infection;
At Step 1306, production of cleaning and disinfection protocols based on the identified microbial infection; and
At Step 1307, transmission of said alerts and cleaning protocols to the facility cleaning and disinfection crews.

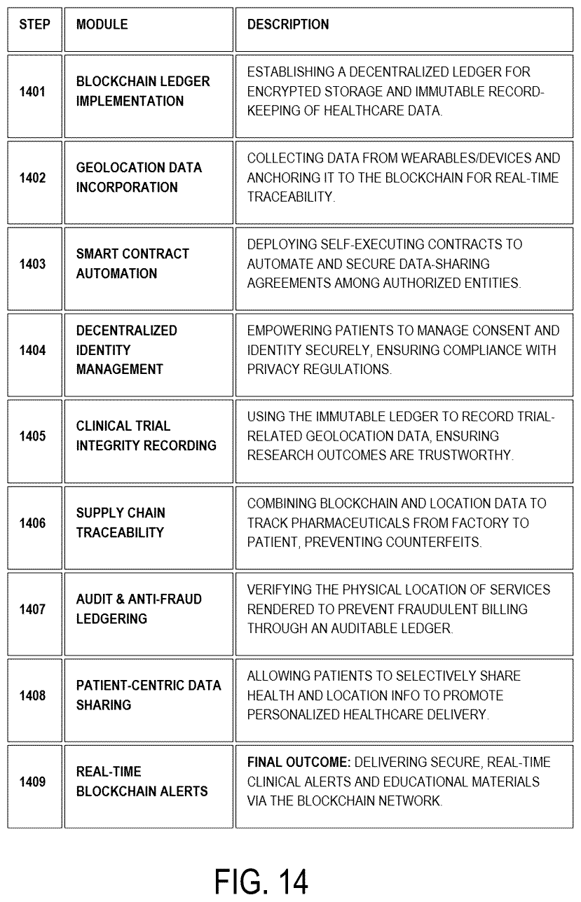

As shown in FIG. 14, some embodiments of the present invention include modules directed to enhancing security, interoperability, and transparency in healthcare data management, wherein a controller, such as a cloud-based server, may implement one or more of the following method steps:
At Step 1401, implementing a blockchain-based decentralized ledger system for securely storing healthcare data, including geolocation information, ensuring cryptographic encryption for each transaction, and leveraging blockchain's inherent immutability to maintain data integrity;
At Step 1402, collecting and analyzing geolocation data from diverse sources, including wearables, medical devices, and patient records, and incorporating said geolocation data into the blockchain for real-time tracking and traceability;
At Step 1403, employing a smart contract module, which, in some embodiments, may record smart contracts on a blockchain (or other shared ledger device) to automate and secure data sharing agreements, to enable seamless and controlled sharing of geolocation and healthcare data among authorized entities based on predefined rules encoded within said smart contracts;
At Step 1404, implementing a decentralized identity management system on the blockchain, allowing patients to control and manage consent for sharing specific geolocation information securely with healthcare providers or researchers while ensuring compliance with privacy regulations;
At Step 1405, leveraging blockchain's transparent and immutable nature to record geolocation data related to clinical trials and research activities, ensuring the integrity of the data, and facilitating trustworthy outcomes in healthcare research.
At Step 1406, establishing end-to-end traceability in the pharmaceutical supply chain by combining blockchain with geolocation data, enabling the secure tracking of drug movement from manufacturing to distribution, minimizing the risk of counterfeit drugs, and ensuring drug authenticity.
At Step 1407, Using blockchain to create a transparent and auditable ledger of healthcare transactions, incorporating geolocation data to verify the location of healthcare services rendered, thereby preventing fraudulent activities in healthcare billing processes.
At Step 1408, enhancing patient-centric healthcare by empowering patients to share their geolocation data and health information selectively and securely with healthcare providers, promoting a personalized approach to healthcare delivery.
At Step 1409, integrating real-time alerts and notifications within the blockchain system to provide healthcare providers with timely insights, personalized prescription recommendations, and educational materials based on geolocation and healthcare data analytics.

Some implementations include cryptographic encryption in the blockchain-based decentralized ledger system ensures the confidentiality and integrity of healthcare data, including geolocation information, protecting patient privacy, and preventing unauthorized access.

Some implementations include using geolocation data from wearables and medical devices to track patient movements and activities, integrating said data into the blockchain to create a comprehensive record of patient health behavior for personalized healthcare interventions.

Some implementations include smart contracts to facilitate dynamic and conditional access controls for geolocation and healthcare data sharing, allowing patients to set granular permissions and preferences for data access by different healthcare entities.

Some implementations include utilizing a decentralized identity management system to enable patients to revoke or modify consent for sharing geolocation information with healthcare entities in real-time, providing patients with control over their data sharing preferences.

Some implementations include using blockchain's transparent and immutable nature to leveraged to create an audit trail for geolocation data in clinical trials, ensuring the credibility of research outcomes and compliance with regulatory standards.

Some implementations include applying geolocation data in the blockchain to monitor and enforce compliance with regulatory standards in the pharmaceutical supply chain, providing transparency and accountability in drug manufacturing, distribution, and authenticity.

Some implementations include real-time alerts and notifications to provide healthcare providers with insights into patient conditions, potential outbreaks, or critical events based on geolocation and healthcare data analytics, facilitating proactive healthcare decision-making.

Some implementations include employing blockchain's timestamping capabilities to record the timing and sequence of healthcare transactions, ensuring the chronological accuracy of geolocation and health data entries.

Some implementations include real-time alerts and notifications to provide actionable recommendations for healthcare providers based on geolocation and healthcare data analytics, enhancing the effectiveness of interventions, and improving patient outcomes.

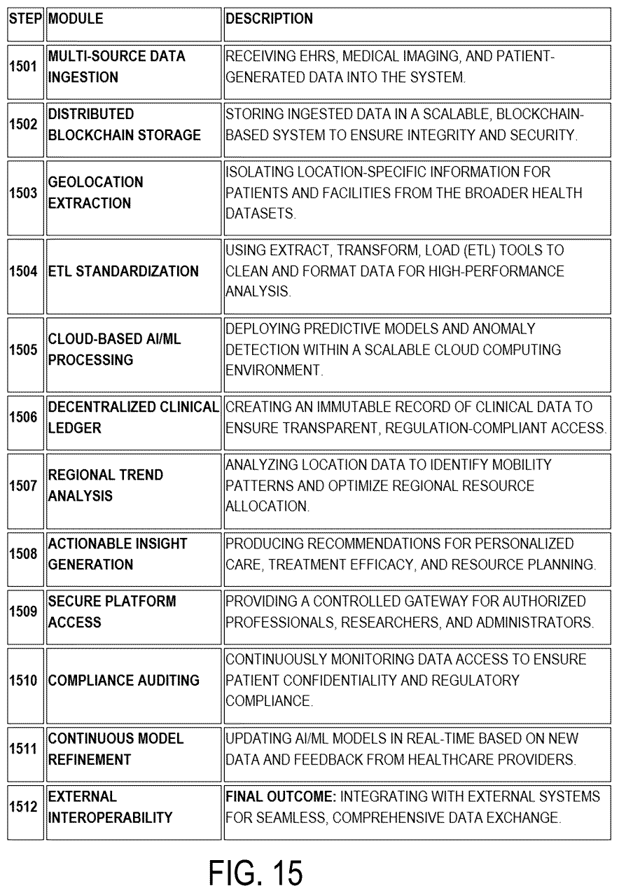

As shown in FIG. 15, some embodiments of the present invention include modules directed integrated healthcare data analytics using a technology stack, a controller, such as a cloud-based server, may implement one or more of the following method steps:
At Step 1501, receiving healthcare data from multiple sources, including electronic health records (EHRs), medical imaging data, and patient-generated data;
At Step 1502, storing said healthcare data in a distributed and scalable storage system, utilizing a blockchain-based storage solution for ensuring data integrity, traceability, and security;
At Step 1503, extracting geolocation information associated with patients and healthcare facilities from said healthcare data;
At Step 1504, employing Extract, Transform, Load (ETL) tools to clean, integrate, and transform the healthcare data into a standardized format suitable for analysis;
At Step 1505, utilizing a cloud-based computing environment for scalable processing of the standardized healthcare data, further comprising deploying AI/ML algorithms and artificial intelligence models for predictive analytics, anomaly detection, and clinical decision support;
At Step 1506, incorporating blockchain technology to create a decentralized and immutable ledger for storing clinical data, ensuring secure and transparent access to patient records while maintaining compliance with healthcare regulations;
At Step 1507, analyzing geolocation data to identify regional health trends, patient mobility patterns, and resource allocation optimization;
At Step 1508, generating actionable insights and recommendations based on the results of the AI/ML data analytics, wherein said insights contribute to personalized patient care, treatment efficacy assessments, and healthcare resource planning;
At Step 1509, providing secure access to the integrated healthcare analytics platform for authorized healthcare professionals, researchers, and administrators;
At Step 1510, auditing and monitoring access to healthcare data, ensuring compliance with data privacy regulations and maintaining patient confidentiality;
At Step 1511, continuously updating and refining the AI/ML models based on real-time healthcare data and feedback from healthcare professionals; and
At Step 1512, facilitating interoperability with external healthcare systems and data sources, allowing seamless integration of additional datasets for comprehensive analytics.

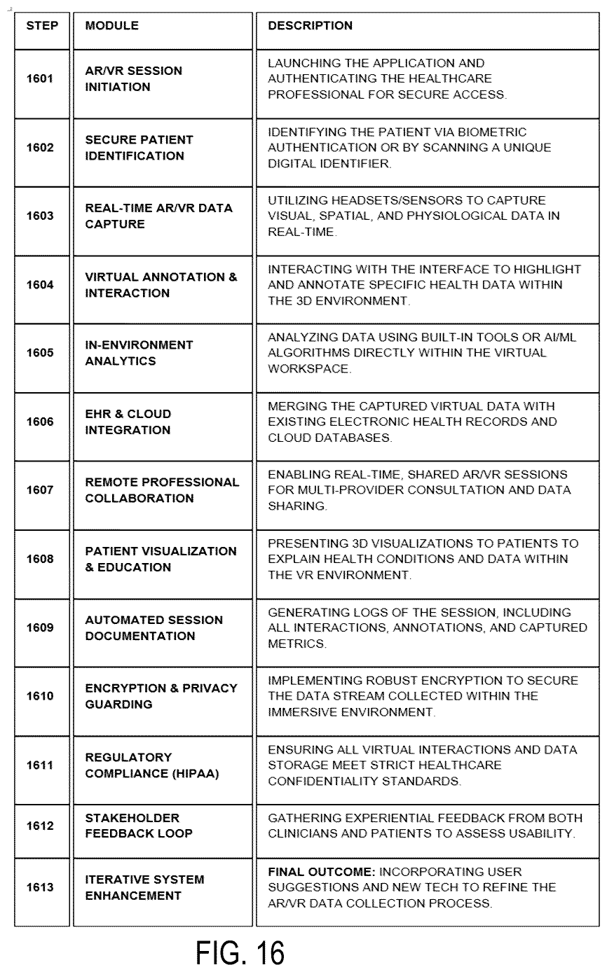

As shown in FIG. 16, some embodiments of the present invention include modules directed to utilizing Augmented Reality (AR) and Virtual Reality (VR) technology to collect patient data to be integrated in a clinical decision support platform, a controller, such as a cloud-based server, may implement one or more of the following method steps:
At Step 1601, initiating an AR/VR application and authenticating a healthcare professional for accessing patient data;
At Step 1602, identifying the patient through secure authentication methods or by scanning a unique patient identifier;
At Step 1603, employing AR/VR technology to capture patient data in real-time, including visual, spatial, and sensor data;
At Step 1604, interacting with the AR/VR interface to annotate and highlight relevant patient data within the virtual environment;
At Step 1605, analyzing the collected patient data within the AR/VR environment using built-in analytics tools or integrated AI/ML algorithms;
At Step 1606, integrating the analyzed data with existing electronic health records (EHR) systems or cloud-based databases for comprehensive patient management;
At Step 1607, enabling real-time collaboration between healthcare professionals by sharing the AR/VR session or data annotations;
At Step 1608, facilitating communication with patients by presenting visualizations and explanations of their health data within the AR/VR environment;
At Step 1609, automatically generating documentation of the AR/VR session, including captured data, annotations, and interactions;
At Step 1610, implementing robust encryption and authentication mechanisms to ensure the security and privacy of patient data collected within the AR/VR environment;
At Step 1611, complying with relevant healthcare regulations, such as HIPAA, to protect patient confidentiality and data integrity;
At Step 1612, gathering feedback from healthcare professionals and patients to continuously improve the usability, functionality, and effectiveness of the AR/VR application for data collection; and
At Step 1613, incorporating user suggestions and technological advancements to enhance the AR/VR data collection process over time.

In some implementations, an AR/VR application is utilized in a hospital setting for bedside patient care, surgical planning, or medical training.

In some implementations, a unique patient identifier is generated by the healthcare facility's electronic health record (EHR) system and linked to the patient's medical record.

In some implementations, visual data captured includes medical imaging modalities such as X-ray, CT scan, MRI, or ultrasound images overlaid onto the AR/VR environment for enhanced visualization and interpretation.

In some implementations, spatial data captured comprises a virtual representation of the patient's surgical site, allowing surgeons to perform virtual preoperative planning and rehearsal of complex surgical procedures.

In some implementations, sensor data captured includes continuous monitoring of patient vital signs during surgery or remote patient monitoring in intensive care units (ICUs), enabling early detection of clinical deterioration.

In some implementations, annotations and highlights applied to the patient data within the AR/VR environment include anatomical landmarks, pathological findings, or surgical landmarks for improved surgical precision and communication among surgical team members.

In some implementations, analytics tools used for data analysis within the AR/VR environment include predictive analytics algorithms for early detection of sepsis, deterioration in patient condition, or adverse events during surgery.

In some implementations, integration of analyzed data with existing electronic health records (EHR) systems or cloud-based databases enables automatic documentation of surgical procedures, intraoperative findings, and postoperative outcomes for quality improvement and research purposes.

In some implementations, real-time collaboration between healthcare professionals includes multidisciplinary team meetings conducted in virtual reality environments to review complex patient cases, discuss treatment plans, and make clinical decisions.

In some implementations, communication with patients within the AR/VR environment includes providing immersive educational content about their medical condition, treatment options, and self-care instructions to improve health literacy and patient engagement.

In some implementations, documentation of the AR/VR session includes video recordings of surgical procedures, annotated screenshots of medical imaging studies, and electronic signatures of healthcare providers for legal and regulatory compliance.

In some implementations, encryption and authentication mechanisms may be used to secure patient data within the AR/VR environment comply with healthcare privacy regulations such as the Health Insurance Portability and Accountability Act (HIPAA) to protect patient confidentiality and prevent unauthorized access.

In some implementations, feedback gathered from healthcare professionals and patients may be used to refine the AR/VR application's functionalities for specific clinical workflows, specialty areas, and patient populations to optimize usability and effectiveness in healthcare delivery.

In some implementations, authentication of a healthcare professional may include biometric authentication, password authentication, or token-based authentication.

In some implementations, a unique patient identifier may be embodied in a barcode, QR code, RFID tag, or patient-specific identifier generated by the healthcare facility.

In some implementations, visual data captured may include medical images, diagnostic reports, and patient demographics overlaid onto the AR/VR environment.

In some implementations, spatial data captured may include a three-dimensional representation of the patient's anatomy, allowing for detailed examination and manipulation.

In some implementations, sensor data captured may include physiological parameters such as heart rate, blood pressure, temperature, and oxygen saturation measured using sensors integrated into the AR/VR environment or external medical devices.

In some implementations, annotations and highlights applied to the patient data within the AR/VR environment include text annotations, graphical annotations, or voice annotations.

In some implementations, analytics tools used for data analysis within the AR/VR environment include statistical analysis, pattern recognition algorithms, or predictive modeling techniques.

In some implementations, integration of analyzed data with existing electronic health records (EHR) systems or cloud-based databases is facilitated through standard healthcare interoperability protocols such as HL7 (Health Level Seven) or FHIR (Fast Healthcare Interoperability Resources) in data formats such as JSON.

In some implementations, real-time collaboration between healthcare professionals includes simultaneous viewing and manipulation of patient data within the AR/VR environment by multiple users.

In some implementations, communication with patients within the AR/VR environment includes providing interactive educational content, treatment explanations, or virtual consultations with healthcare providers.

In some implementations, documentation of the AR/VR session includes timestamps, user interactions, and data snapshots captured during the session for audit and review purposes.

In some implementations, encryption and authentication mechanisms used to secure patient data within the AR/VR environment comply with industry standards such as AES (Advanced Encryption Standard) and RSA (Rivest-Shamir-Adleman).

In some implementations, feedback gathered from healthcare professionals and patients is used to iteratively improve the user interface, data visualization tools, and data analysis algorithms of the AR/VR application.

In some implementations, user suggestions and technological advancements incorporated into the AR/VR application include new features, improved performance, and compatibility with emerging AR/VR hardware platforms.

In some implementations, integrating geolocation tracking functionality into the AR/VR application may be used to capture and annotate patient data based on the physical location of the healthcare professional within a healthcare facility, wherein the geolocation data is utilized to enhance context-awareness and streamline data collection workflows.

In some embodiments, the present invention encompasses a comprehensive approach to alerting and guiding facility cleaning and disinfection crews in response to microbial infections. This multifaceted process involves several key steps to ensure an efficient and targeted response. Firstly, the method involves the collection of microbial data from designated rooms within a facility. Utilizing advanced sensors, cameras, or other data collection devices, the system gathers real-time information to identify the presence of various microorganisms. Subsequently, the collected microbial data undergoes a rigorous analysis using state-of-the-art AI/ML algorithms. This analytical process is designed to identify specific microbial infections with a high degree of accuracy. The system is trained on a diverse dataset, enhancing its capability to recognize a wide range of microorganisms.

Upon successful identification, the method automatically generates alerts that provide detailed information about the type of microbial infection detected. These alerts serve as helpful notifications for the cleaning and disinfection crews, allowing them to prepare for targeted interventions. An innovative aspect of this method involves the intelligent selection of the appropriate cleaning and disinfecting product tailored to the identified microbial infection. The system takes into consideration the nature and severity of the infection, ensuring that the crews utilize the most effective products for eradication. Furthermore, the method includes the production of cleaning and disinfection protocols based on the identified microbial infection. These protocols are dynamically generated, considering the specific characteristics of the detected microorganisms. This ensures that the cleaning procedures are not only effective but also tailored to the unique requirements of each situation. Lastly, the method emphasizes the importance of seamless communication by facilitating the transmission of the generated alerts and cleaning protocols to the facility cleaning and disinfection crews. This ensures that the information is promptly and efficiently delivered, enabling the crews to initiate the necessary actions promptly. In summary, this method introduces a holistic and intelligent approach to facility cleaning and disinfection, leveraging AI/ML algorithms to enhance the accuracy, efficiency, and customization of microbial infection response protocols.

The integration of blockchain technology with geolocation and advanced data analytics in healthcare settings holds the potential to revolutionize various aspects of the healthcare industry, providing enhanced security, interoperability, and transparency. One of the key advantages of incorporating blockchain is the bolstering of data security and integrity. Blockchain's decentralized and cryptographic features provide a robust solution for securing geolocation and healthcare data. Through encryption, each transaction or data entry is secured, and the decentralized nature ensures that data is distributed across multiple nodes, minimizing the risk of unauthorized access or tampering. The immutability inherent in blockchain ensures that once data is recorded, it becomes resistant to alteration, preserving the integrity of patient geolocation data and health records. Interoperability and seamless data sharing become more achievable with the integration of blockchain, geolocation, and advanced data analytics. Smart contracts, executable code on the blockchain, can facilitate secure and automated data sharing agreements. This is particularly relevant for geolocation data sourced from wearables or medical devices, enabling its smooth exchange among authorized entities based on predefined rules encoded in smart contracts.

Additionally, blockchain serves as a standardized and distributed ledger for healthcare data, fostering interoperability between different systems, with geolocation data easily shared across healthcare providers. Decentralized identity management is another area where blockchain can play a significant role in healthcare. Patients can control and manage consent for sharing specific geolocation information securely with healthcare providers or researchers. Blockchain's decentralized identity solutions empower patients, ensuring compliance with privacy regulations such as GDPR and putting individuals in charge of their health data. In the realm of clinical trials and research, blockchain provides a transparent and immutable record of data. Geolocation data collected during trials can be securely stored on the blockchain, offering researchers confidence in the integrity of the data, and promoting trust in the outcomes of clinical studies. The integration of blockchain, geolocation, and data analytics also has applications in the pharmaceutical supply chain. Blockchain's ability to establish end-to-end traceability can enhance the authenticity and integrity of drugs. Combining geolocation data with blockchain ensures accurate tracking of the movement of pharmaceuticals, from manufacturing facilities through distribution channels to pharmacies, minimizing the risk of counterfeit drugs entering the market. Addressing fraudulent activities in healthcare billing is another area where blockchain can make a substantial impact. By creating a transparent and auditable ledger of transactions, blockchain can prevent fraud in billing processes. Geolocation data plays a role in verifying the location of healthcare services, ensuring accurate billing, and reducing the occurrence of fraudulent claims. Moreover, the integration of these technologies contributes to patient-centric healthcare. Blockchain empowers patients to have greater control over their health data, including geolocation information. Patients can selectively and securely share this data with healthcare providers, fostering a more personalized and patient-centric approach to healthcare delivery. While the potential benefits are substantial, challenges such as scalability, regulatory compliance, and interoperability need to be addressed for the seamless integration of blockchain, geolocation, and advanced data analytics in healthcare. Collaborative efforts among stakeholders and the establishment of industry-wide standards will facilitate successful implementation and realizing the full potential of these transformative technologies in healthcare settings.

Technology stacking for performing big healthcare data analytics involves the strategic selection and integration of various technologies to efficiently process, analyze, and derive insights from large volumes of healthcare data. Technology stacking enables organizations to harness the power of data for better patient care, research, and operational efficiency. At the foundation of the technology stack is a robust data storage system. In big healthcare data analytics, where massive datasets are generated daily, scalable and distributed storage solutions, like Apache Hadoop Distributed File System (HDFS) or cloud-based storage services such as Amazon S3 or Google Cloud Storage, are commonly employed. These systems can handle the vast amounts of structured and unstructured data generated by electronic health records (EHRs), medical imaging, and other sources. On top of the data storage layer, a powerful data processing framework is essential. Apache Spark is a popular choice for its ability to handle large-scale data processing tasks in-memory, providing significant speed improvements over traditional MapReduce. Spark allows healthcare organizations to perform complex data transformations and analytics on their massive datasets efficiently. For data cleaning, integration, and transformation tasks, Extract, Transform, Load (ETL) tools play a critical role. Tools like Apache NiFi or Talend help streamline the process of ingesting data from various sources, cleaning and transforming it into a suitable format for analysis. These tools ensure data quality and consistency, which are helpful for accurate healthcare analytics.

With prepared data, processes on the analytics layer comes into play. Data analytics platforms such as Apache Flink or Apache Hadoop are utilized to perform advanced analytics, including AI/ML algorithms for predictive modeling, clustering, and anomaly detection. These technologies enable healthcare professionals to gain insights into patient outcomes, disease patterns, and treatment efficacy. Cloud platforms such as, for example, one or more of: AWS, Azure, or Google Cloud offer scalable computing resources, allowing healthcare organizations to process and analyze data without the need for significant upfront infrastructure investments. Additionally, cloud services provide advanced tools and services for AI/ML, making it easier for healthcare professionals to leverage predictive analytics.

Security and compliance are important in healthcare, and thus, technologies for data security and privacy must be integrated into the stack. Encryption, access controls, and auditing mechanisms ensure that sensitive patient information remains confidential and complies with healthcare regulations such as the Health Insurance Portability and Accountability Act (HIPAA). In conclusion, the technology stack for big healthcare data analytics involves a comprehensive approach, incorporating storage, processing, analytics, and security components. By carefully selecting and integrating these technologies, healthcare organizations can unlock the full potential of their data, driving improvements in patient care, research, and operational efficiency.

Real-time data analytics may be operative across various domains, and the application of AI/ML algorithms may be instrumental in extracting meaningful insights from data, including streaming data.

One category of algorithms used for real-time data analytics includes streaming clustering algorithms, such as, for example, an adaptation of traditional K-Means clustering of continuously evolving datasets. This ensures that clusters are dynamically assigned in real-time, accommodating changes in data distribution as new information becomes available. Online learning algorithms, such as Stochastic Gradient Descent (SGD), enable incremental updates of model parameters with each new data point. This adaptability makes them suitable for scenarios where models need to evolve in real-time. Similarly, online Random Forests extend the concept of traditional Random Forests to accommodate changing data streams, ensuring continuous learning and adaptation. Anomaly detection algorithms play a helpful role in real-time analytics, with the Isolation Forest algorithm identifying anomalies by isolating them into small clusters. This approach is effective for real-time detection of unusual patterns. Additionally, One-Class SVM is adept at learning normal instances and detecting anomalies, making it suitable for real-time anomaly detection scenarios.

Sequential pattern mining algorithms, such as, for example, the Apriori Algorithm, may be employed for real-time association rule mining. These algorithms identify patterns in sequential data, making them valuable for applications where understanding temporal relationships is important. Time series forecasting algorithms are pivotal in real-time analytics, and examples include ARIMA for time-series forecasting with trend and seasonality, and Prophet, a model developed by Facebook, designed for forecasting with daily observations displaying patterns on different time scales. Reinforcement learning algorithms, such as the Deep Q-Network (DQN), are applied to real-time decision-making scenarios.

Reinforcement learning algorithms learn optimal strategies through trial and error, adapting their decision-making processes based on real-time feedback. Natural Language Processing (NLP) algorithms contribute significantly to real-time analytics. Word embeddings like Word2Vec and GloVe are employed for semantic analysis of text data, while advanced models like BERT enable real-time natural language understanding and sentiment analysis. Deep learning models, including Recurrent Neural Networks (RNN) and Long Short-Term Memory (LSTM) networks, are helpful for real-time analytics, particularly in tasks involving sequential data. These models excel in tasks such as language modeling and time-series analysis, capturing complex dependencies in real-time data. Ensemble learning algorithms, exemplified by Adaptive Boosting (AdaBoost), are adaptable for real-time classification tasks. These algorithms combine predictions from multiple weak learners, enhancing the robustness of real-time classification models. Finally, online decision trees like the Hoeffding Tree are designed for real-time classification in data streams. These trees adapt to changing patterns and evolving data, making them suitable for dynamic real-time analytics applications. The choice of algorithm depends on the specific characteristics of the data and the objectives of the real-time analytics application, showcasing the versatility and adaptability of AI/ML in extracting insights from streaming data.

According to the present invention, Virtual Reality (VR) and Augmented Reality (AR) may be deployed in data applications and real-time clinical decision-making platforms. AR and VR technologies offer immersive experiences that can revolutionize various aspects of healthcare delivery. In training and education, VR and AR simulations replicate medical procedures and anatomical structures, providing invaluable learning experiences for medical students and practitioners. Data from these simulations can be seamlessly integrated into training platforms to monitor progress and personalize learning experiences.

Additionally, health care practitioners, such as surgeons, may utilize VR and AR for precise surgical planning by visualizing patient anatomy in three dimensions, thereby enhancing surgical outcomes. Patient education and engagement are also transformed, as VR and AR applications facilitate a better understanding of medical conditions and treatment options, leading to improved adherence and health outcomes.

Furthermore, remote consultations and telemedicine are facilitated through VR and AR technologies, allowing for real-time interaction and data sharing regardless of geographical distance. According to the present invention, “real time” may include no artificial time delays. Rehabilitation and therapy programs can be personalized through VR, with real-time data tracking enabling therapists to optimize treatment plans and monitor progress effectively. Integration of VR and AR data applications into clinical decision support systems (CDSS) enhances clinicians' capabilities by providing immersive visualizations of patient data, aiding in accurate diagnoses and treatment selection.

Moreover, VR and AR technologies support medical research and development endeavors by enabling data visualization, simulation-based experiments, and collaborative work environments for researchers. Integration into real-time clinical decision platforms necessitates interoperability with existing healthcare systems, adherence to data privacy regulations, and advanced analytics capabilities to provide intelligent insights and decision support. User-friendly interfaces and ergonomic design considerations are vital for ensuring the adoption and usability of VR and AR technologies in clinical settings, ultimately enhancing the efficiency and quality of healthcare delivery.

According to the present invention, real-time data integration platforms harmoniously combine geolocation, blockchain, patient data, clinical decision support, and advanced data analytics are vast and transformative across various sectors. One primary application includes healthcare management, where real-time integration facilitates efficient patient tracking and monitoring.

The present invention provides platforms that enable healthcare providers to access up-to-the-minute patient data, allowing for personalized care plans, timely interventions, and streamlined communication among medical staff. The platform enhances emergency response by optimizing ambulance deployment through real-time geolocation data. Quick access to patient information aids in routing ambulances to the nearest and most suitable healthcare facilities, potentially saving critical time in emergencies. Real-time data integration plays a crucial role in infectious disease control. By utilizing blockchain for secure data sharing, healthcare authorities can track and analyze the spread of diseases in real-time, enabling early detection, rapid response, and effective prevention strategies. Clinical decision-making receives a significant boost with real-time analytics and decision support. Healthcare providers can access personalized treatment plans based on the latest patient data, ensuring informed and optimized care strategies.

The present invention provides for a computing platform, such as a cloud-based Software as a Service (SaaS) streamlines clinical trials and research endeavors. Researchers can leverage real-time patient data for efficient participant recruitment, continuous monitoring, and quicker insights into trial progress, ultimately accelerating the pace of medical research. For chronic disease management, continuous real-time monitoring empowers healthcare providers to proactively intervene. The platform's capabilities enable personalized care plans, medication adherence monitoring, and early identification of potential issues. In the realm of nephrological conditions, the platform enhances the management of chronic kidney disease. Real-time monitoring of patient data facilitates better understanding of treatment outcomes, medication adherence, and overall kidney health. The platform aids in post-surgical monitoring, helping identify complications in real-time and preventing potential sepsis risks. Timely interventions based on real-time analytics contribute to reducing hospital readmissions and improving post-surgical outcomes.

Telemedicine and remote patient monitoring benefit from continuous geolocation tracking. The platform enables healthcare providers to remotely monitor patients, ensuring ongoing care and intervention, particularly valuable for those in remote locations. Geofencing adds an extra layer of security for patient safety. Sensitive medical facilities can implement geofencing, restricting access to authorized personnel, and enhancing overall security protocols. Real-time data analytics contribute to effective population health management. By monitoring disease trends in real-time, healthcare authorities can implement timely interventions, allocate resources efficiently, and enhance overall public health.

In healthcare facilities, the platform contributes to optimized asset and inventory management using blockchain technology. It ensures the secure and efficient utilization of healthcare resources, reducing operational costs and enhancing overall efficiency. In essence, the real-time data integration platform redefines the landscape of healthcare by providing a holistic and dynamic approach to data utilization. Its applications extend far beyond traditional healthcare management, influencing emergency response, disease control, research, and various specialized medical domains. The platform emerges as a catalyst for innovation, efficiency, and improved patient outcomes in the evolving healthcare landscape.

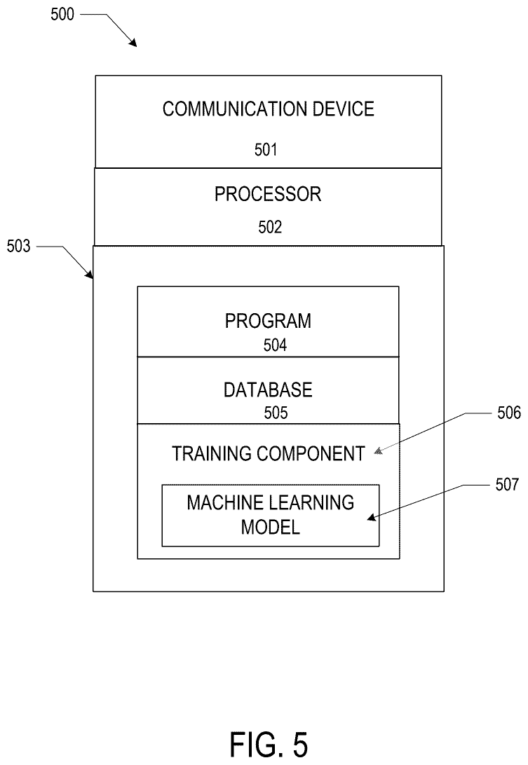

Referring now to FIG. 5, an automated controller is illustrated that may be used to implement various aspects of the present disclosure, in various embodiments, and for various aspects of the present disclosure, controller 500 may be included in one or more of: a wireless tablet or handheld device, a server, a rack mounted processor unit. The controller may be included in one or more of the apparatuses described above, such as a Server, and a Network Access Device. The controller 500 includes a processor unit 502, such as one or more semiconductor-based processors, coupled to a communication device 501 configured to communicate via a communication network (not shown in FIG. 5). The communication device 501 may be used to communicate, for example, with one or more online devices, such as a personal computer, laptop, or a handheld device.

The processor 502 is also in communication with a storage device 503. The storage device 503 may comprise any appropriate information storage device, including combinations of magnetic storage devices (e.g., magnetic tape and hard disk drives), optical storage devices, and/or semiconductor memory devices such as Random Access Memory (RAM) devices and Read Only Memory (ROM) devices.

The storage device 503 can store a software program 504 with executable logic for controlling the processor 502. The processor 502 performs instructions of the software program 504, and thereby operates in accordance with the present disclosure. In some embodiments, the processor may be supplemented with a specialized processor for AI related processing. The processor 502 may also cause the communication device 501 to transmit information, including, in some instances, control commands to operate apparatus to implement the processes described above. The storage device 503 can additionally store related data in a database 505. The processor and storage devices may access an AI training component 506 and database, as needed, which may also include storage of machine learned models 507.

A mobile device may include an optical capture device to capture an image and convert it to machine-compatible data, and an optical path, typically a lens, an aperture, or an image conduit to convey the image from the rendered document to the optical capture device. The optical capture device may incorporate a Charge-Coupled Device (CCD), a Complementary Metal Oxide Semiconductor (CMOS) imaging device, or an optical Sensor of another type.

A microphone and associated circuitry may convert the sound of the environment, including spoken words, into machine-compatible signals. Input facilities may exist in the form of buttons, scroll wheels, or other tactile Sensors such as touchpads. In some embodiments, input facilities may include a touchscreen display.

Visual feedback to the user is possible through a visual display, touchscreen display, or indicator lights. Audible feedback may come from a loudspeaker or other audio transducer. Tactile feedback may come from a vibrate module.

A motion Sensor and associated circuitry convert the motion of the mobile device into machine-compatible signals. The motion Sensor may comprise an accelerometer that may be used to sense measurable physical acceleration, orientation, vibration, and other movements. In some embodiments, motion Sensor may include a gyroscope or other device to sense different motions.

A location Sensor and associated circuitry may be used to determine the location of the device. The location Sensor may detect Global Position System (GPS) radio signals from satellites or may also use assisted GPS where the mobile device may use a cellular network to decrease the time necessary to determine location.

The mobile device comprises logic to interact with the various other components, possibly processing the received signals into different formats and/or interpretations. Logic may be operable to read and write data and program instructions stored in associated storage or memory such as RAM, ROM, flash, or other suitable memory. It may read a time signal from the clock unit. In some embodiments, the mobile device may have an on-board power supply. In other embodiments, the mobile device may be powered from a tethered connection to another device, such as a Universal Serial Bus (USB) connection.

The mobile device also includes a network interface to communicate data to a network and/or an associated computing device. Network interface may provide two-way data communication. For example, network interface may operate according to the internet protocol. As another example, network interface may be a local area network (LAN) card allowing a data communication connection to a compatible LAN. As another example, network interface may be a cellular antenna and associated circuitry which may allow the mobile device to communicate over standard wireless data communication networks. In some implementations, network interface may include a Universal Serial Bus (USB) to supply power or transmit data. In some embodiments, other wireless links may also be implemented.

As an example of one use of mobile device, a reader may scan an input drawing with the mobile device. In some embodiments, the scan may include a bit-mapped image via the optical capture device. Logic causes the bit-mapped image to be stored in memory with an associated timestamp read from the clock unit. Logic may also perform optical character recognition (OCR) or other post-scan processing on the bit-mapped image to convert it to text.

A directional sensor may also be incorporated into the mobile device. The directional device may be a compass and be based upon a magnetic reading or based upon network settings.

From the foregoing, it will be understood that various aspects of the processes described herein are software processes that execute on computer systems that form parts of the system. Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid-state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a general-purpose computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such a connection is properly termed and considered a computer-readable medium. Combinations of the above should also be included within the scope of computer-readable media. Computer-executable instructions comprise, for example, instructions and data which cause a general-purpose computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed innovations may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represents examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed innovation are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language, or other input devices (not shown), such as a microphone, etc. These and other input devices are often connected to the processing unit through known electrical, optical, or wireless connections.

The computer that affects many aspects of the described processes will typically operate in a networked environment using logical connections to one or more remote computers or data sources, which are described further below. Remote computers may be another personal computer, a server, a router, a network PC, a peer device, or other common network node, and typically include many or all of the elements described above relative to the main computer system in which the innovations are embodied. The logical connections between computers include a local area network (LAN), a wide area network (WAN), a global satellite network, virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the innovation is connected to the local network through a network interface or adapter. When used in a WAN or WLAN networking environment, the computer may include a modem, a wireless link, or other mechanisms for establishing communications over the wide-area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed innovations will be readily discernible from the description herein, by those of ordinary skill in the art. Many embodiments and adaptations of the disclosure and claimed innovations other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed innovations. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed innovations. In addition, some steps may be carried out simultaneously, contemporaneously, or in synchronization with other steps.

The embodiments were chosen and described in order to explain the principles of the claimed innovations and their practical application so as to enable others skilled in the art to utilize the innovations and various embodiments and with various modifications as are suited to the particular use contemplated. Alternative embodiments will become apparent to those skilled in the art to which the claimed innovations pertain without departing from their spirit and scope. Accordingly, the scope of the claimed innovations is defined by the appended claims rather than the foregoing description and the exemplary embodiments described therein.

The present invention provides for systems of one or more computers that can be configured to perform particular operations or actions by virtue of having software, firmware, hardware, or a combination of them installed on the system that in operation causes or cause the system to perform the actions. One or more computer programs can be configured to perform artificial intelligence operations or actions by virtue of including instructions that, when executed by data processing apparatus, cause the apparatus to perform the actions.

### Conclusion

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood that the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” be used in a permissive sense (i.e., meaning having the potential to), rather than the mandatory sense (i.e., meaning must). Similarly, the words “include,” “including,” and “includes” mean including but not limited to. To facilitate understanding, like reference numerals have been used, where possible, to designate like elements common to the figures.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising,” “including,” and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

1. An apparatus for integrated real-time healthcare data management with geolocation capabilities, comprising:
a controller comprising a processor in logical communication with a digital storage device storing executable software, the executable software is executable upon command to cause the processor to:
receive geolocation data from a plurality of sources;
generate harmonized patient data from data received into the controller from various sources;
provide evidence-based insights and recommendations; and
analyze the harmonized patient data and generate actionable insights;
a security device for maintaining integrity of the harmonized patient data; and
a smart contract module configured to automate data sharing agreements and enable controlled access to geolocation and healthcare data among authorized entities.

2. The apparatus of claim 1, additionally comprising a geolocation mechanism operative to receive the data from one or more of: a GPS tracker, WiFi triangulation, cellular triangulation, Bluetooth beacon, and near-field communication device, and determine a geolocation.

3. The apparatus of claim 1, wherein a data harmonization module integrates data from Electronic Health Records (EHR), Remote Patient Monitoring, physician offices, imaging, nursing homes, home care, clinical labs, point-of-care devices, Internet of Things (IoT) devices, and smart wearables.

4. The apparatus of claim 1, wherein the security device comprises a blockchain-based decentralized ledger and cryptographic encryption.

5. The apparatus of claim 1, wherein a clinical decision support platform incorporates AI/ML algorithms to provide personalized recommendations based on patient-specific data and geolocation information.

6. The apparatus of claim 1, wherein an advanced data analytics module performs predictive modeling, trend analysis, and anomaly detection to identify meaningful patterns and correlations within the harmonized patient data.

7. The apparatus of claim 1, further wherein the smart contract module is configured to record smart contracts on a blockchain to secure data included in the smart contracts.

8. The apparatus of claim 1, further comprising a patient consent management module that allows individuals to manage consent for sharing specific geolocation information.

9. The apparatus of claim 5, wherein the clinical decision support platform is configured to adapt its recommendations based on a current location of a patient and local healthcare resources.

10. The apparatus of claim 6, wherein the advanced data analytics module is configured to identify geographical health disparities and optimize resource allocation based on the geolocation data.

11. The apparatus of claim 5, wherein the clinical decision support platform is configured to provide real-time alerts and notifications to healthcare providers based on geolocation patterns and clinical data.

12. The apparatus of claim 1, further comprising a telemedicine module configured to facilitate remote patient monitoring and virtual consultations based on the geolocation data.

13. The apparatus of claim 5, wherein the clinical decision support platform is configured to integrate data from wearable devices, including smart rings, shirts, belts, glasses, and contacts, to enhance accuracy of patient location tracking.

14. A method for integrated real-time healthcare data management with geolocation capabilities, the method comprising the steps of:
receiving geolocation data from a plurality of sources;
harmonizing patient data from various sources;
securely storing the harmonized patient data on a blockchain-based decentralized ledger;
providing evidence-based insights and recommendations through a clinical decision support platform; and
analyzing the harmonized patient data using advanced data analytics to generate actionable insights.

15. The method of claim 14, further comprising incorporating AI/ML algorithms in the clinical decision support platform to provide personalized recommendations based on patient-specific data and geolocation information.

16. The method of claim 14, further comprising performing predictive modeling, trend analysis, and anomaly detection to identify meaningful patterns and correlations within the harmonized patient data using the advanced data analytics.

17. The method of claim 14, further comprising adapting the recommendations based on a patient's current location and local healthcare resources through the clinical decision support platform.

18. The method of claim 14, further comprising providing real-time alerts and notifications to healthcare providers based on geolocation patterns and clinical data through the clinical decision support platform.

19. The method of claim 14, further comprising performing longitudinal data analysis to understand patient health trends and assess an effectiveness of treatments over time using the advanced data analytics.

20. A system for integrated real-time healthcare data management with geolocation capabilities, comprising: a controller including at least one processor and a memory storing executable instructions that, when executed, cause the controller to operate a geolocation ingestion interface configured to receive geolocation data from a plurality of sources including GPS trackers, Wi-Fi triangulation, cellular triangulation, Bluetooth beacons, near-field communication devices, login credentials, and check-ins; an application programming interface configured to receive device-sourced patient data including patient data files and raw IoT data from one or more of patients, providers, devices, third parties, and applications;
a data harmonization module configured to integrate and standardize patient data from Electronic Health Records, Remote Patient Monitoring systems, physician offices, imaging systems, nursing homes, home care services, clinical laboratories, point-of-care devices, Internet of Things devices, and smart wearables including rings, shirts, belts, glasses, and contacts;
an anonymization and deanonymization service configured to selectively include or exclude personally identifiable information within the harmonized patient data under access policies;
a decentralized ledger configured to securely store the harmonized patient data with cryptographic encryption, immutability, and traceable transaction records;
a Continuous Analytics Processing System comprising a file repository, a transform component, working databases, analyzers, and input/output results modules operative to produce analysis artifacts and dashboards;
a job management subsystem configured to manage partner data, client data, event data, and system logs;
a clinical decision support platform operative on the controller and configured to generate evidence-based insights, treatment recommendations, personalized guidelines, and real-time alerts based on patient-specific data and geolocation context;
an advanced data analytics engine configured to perform descriptive analytics, geospatial analysis using polygons and polygon references, proximity analysis, facial recognition for identity verification and access tracking, network analysis over Bluetooth, NFC, and Wi-Fi interaction graphs, temporal analysis of time-stamped clinical and location events, predictive analytics for disease outbreak risk, patient deterioration, and resource allocation, classification and clustering to assign the one or more patients to risk groups and cohort segments, anomaly detection for abnormal behaviors, security events, and metric deviations, natural language processing to extract structured insights from EHR notes and clinician or patient communications, regression analysis to estimate outcomes using clinical, IoT, and wearable features, and longitudinal analysis to assess patient health trends and treatment effectiveness over time;
a secure cloud subsystem in communication with the controller and configured to host executable code for the file repository, data transformations, analyzers, and storage of Electronic Health Record events and Remote Patient Monitoring events;
an edge deployment facility configured to host controllers and storage proximate to care facilities; and
a smart device application presenting a CAPS dashboard and CAPS application, wherein the system is further configured to generate operational outputs including clinical and hospital-level insights, time-series risk trends, and monitoring tools, and to implement geolocation-aware interventions including geofenced alerts, targeted testing, and resource redeployment to assist infection prevention and containment based on analysis of harmonized geolocation and clinical data.
